(19) United States
(12) Patent Application Publication
(10) Pub. No.: US 2007/0123884 A1
(43) Pub. Date: May 31, 2007

(54) Bone fixation systems and methods of implantation

(76) Inventor: M.S. Abdou, San Diego, CA (US)

Correspondence Address: FISH & RICHARDSON, PC, P.O. BOX 1022, MINNEAPOLIS, MN, 55440-1022, US

(21) Appl. No.: 11/595,801

(22) Filed: Nov. 9, 2006

Related U.S. Application Data
(60) Provisional application No. 60/734,842, filed on Nov. 9, 2005.
(60) Provisional application No. 60/756,081, filed on Jan. 4, 2006.
(60) Provisional application No. 60/757,828, filed on Jan. 10, 2006.
(60) Provisional application No. 60/761,843, filed on Jan. 25, 2006.

(51) Int. Cl. A61F 2/30

(52) U.S. Cl. 606/69

(57) ABSTRACT

A bone plate system is adapted to be attached to bone. The system includes a base plate having at least one aperture for location adjacent to a bone member and a bone screw sized to be inserted through the aperture such that the bone screw sits within a seat of the aperture for engaging the bone member. A retainer member is adapted to transition between a deployed state wherein the retainer member at least partially blocks the aperture for retaining the bone screw in the seat, and an undeployed state wherein the bone screw permits the bone screw to be inserted into the aperture. The retainer member automatically transitions to the undeployed state as the bone screw is inserted into the aperture and automatically transitions to the deployed state once the bone screw is seated within the seat.

### REFERENCE TO PRIORITY DOCUMENT

This application claims priority of the following co-pending U.S. Provisional Patent Applications: (1) U.S. Provisional Patent Application Ser. No. 60/734,842, filed Nov. 9, 2005; (2) U.S. Provisional Patent Application Ser. No. 60/756,081, filed Jan. 4, 2006; (3) U.S. Provisional Patent Application Ser. No. 60/757,828, filed Jan. 10, 2006; and (4) U.S. Provisional Patent Application Ser. No. 60/761,843, filed Jan. 25, 2006. Priority of the aforementioned filing dates is hereby claimed and the disclosures of the Provisional Patent Applications are hereby incorporated by reference in their entirety.

### BACKGROUND

The present disclosure is directed at skeletal plating systems, components thereof, and methods of implant sizing and implant placement. These systems are used to adjust, align and maintain the spatial relationship(s) of adjacent bones or bony fragments during healing and fusion after surgical reconstruction of skeletal segments.

Whether for degenerative disease, traumatic disruption, infection or neoplastic invasion, surgical reconstructions of the bony skeleton are common procedures in current medical practice. Regardless of anatomical region or the specifics of the reconstructive procedure, many surgeons employ an implantable skeletal plate to adjust, align and maintain the spatial relationship(s) of adjacent bones or bony fragments during postoperative healing. These plates are generally attached to the bony elements using bone screws or similar fasteners and act to share the load and support the bone as osteosynthesis progresses.

Available plating systems used to fixate the cervical spine posses several shortcomings. These plates often employ a bone screw retainer in order to reduce the likelihood of screw/plate disconnection and insure that loose screws do not migrate into the cervical soft tissues. The retainer may consist of an additional plate component that covers the bone screw head and prevents its back-out or a friction device that increases screw/plate contact and diminishes the likelihood of screw pullout. With either approach, however, an additional step is required to deploy the locking segment after bone screw placement. This step is often cumbersome, since the locking elements tend to be small in size, difficult to properly position, and contain threads that easily strip with placement.

The implantation procedures of current plates have additional shortcomings. Distraction screws are used during disc removal and subsequent bone work and these screws are removed prior to bone plate placement. The empty bone holes created by removal of the distraction screws can interfere with proper placement of the bone screws used to anchor the plate and predispose to poor plate alignment along the long axis of the spine. This is especially problematic since the surgical steps that precede plate placement will distort the anatomical landmarks required to ensure proper plate alignment, leaving the surgeons with little guidance during plate implantation. For these reasons, bone plates are frequently placed “crooked” in the vertical plane and often predispose to improper bony alignment.

During implantation, there is currently no reliable method to determine the size of the required plate. For this reason, most surgeons make a rough measurement of the grafted level and bring several plates of varying sizes to the operative site. Each of the plates is then placed onto the spine and the appropriate plate size is determined by trail and error. This method of implant size determination is imprecise, inefficient and it unnecessarily lengthens the operative procedure.

Lastly, when bone plates are used to fixate the cervical spine, the plate's midline is placed in the spinal midline so that, at each level, one bone screw is placed on each side of the vertebral midline. Unfortunately, some patients will experience post-operative swallowing difficulties and it is believed that this problem can be minimized by reducing the extent of soft tissue retraction during the plating procedure. Since a left-sided surgical approach requires soft tissue retraction to the right and a right-sided approach necessitates retraction to the left, placement of the bone screws on the side of the plate opposite to that of the side of approach is the step in the procedure that requires the greatest amount of retraction. Thus, soft tissue retraction can be reduced by placing the screws opposite to the surgical approach closer to the vertebral midline.

In view of the proceeding, it would be desirable to design an improved bone plating system and placement protocol. The new device can provide ease of use and reliable bone fixation.

### SUMMARY

In view of the shortcomings of the prior art, various plate embodiments are illustrated to address the foregoing problems. In some embodiments, self-deploying screw-retainer mechanisms are placed at the lower border and/or lateral border of each screw bore hole. Since the midline is not used for retainer placement, this feature advantageously permits one or more midline slots to be incorporated into the plate without increasing the plate width.

In another embodiment, the midline slots accommodate the distraction screws and permit their incorporation into the plate placement protocol. Since the distraction screws are placed early in the surgical procedure, the surgical landmarks are still intact. Use of the distraction screws as a guide for plate placement significantly increases the likelihood of proper placement. In one embodiment, a single segment distraction screws is used while, in another embodiment, a multi-segmental distraction is employed. In the latter, the proximal end of the distraction screw is detached after the bone work is completed. The distal segment is left attached to the vertebra and used to guide the bone plate into the correct placement position. It also serves to immobilize the plate while the plate's bone screws are placed.

In another embodiment, a plate placement instrument is disclosed that advantageously positions the plate at the optimal placement site and then immobilizes it relative to the vertebrae by locking the plate onto a distraction screw. In another embodiment, a method for selection of the optimal plate size is illustrated. Embodiments with varied borehole configurations are disclosed. These devices attempt to minimize extent of post-operative swallowing difficulties by reducing the extent of intra-operative soft tissue retraction.

In one aspect, there is described a bone plate system comprising: a base plate having at least one aperture for location adjacent to a bone member, the aperture including a seat; a bone screw sized to be inserted through the aperture such that the bone screw sits within the seat of the aperture for engaging the bone member; and a retainer member adjacent the aperture, the retainer member adapted to transition between a deployed state wherein the retainer member at least partially blocks the aperture for retaining the bone screw in the seat, and an undeployed state wherein the bone screw permits the bone screw to be inserted into the aperture, wherein the retainer member automatically transitions to the undeployed state as the bone screw is inserted into the aperture and automatically transitions to the deployed state once the bone screw is seated within the seat.

In another aspect, there is described an instrument for placing a bone plate, comprising: a handle; and at least one grasping member that removably attaches to a bone plate to deliver the plate to an operative field wherein the instrument transitions to a locked state wherein at least a portion of the instrument locks to at least one screw attached to underlying bone.

In another aspect, there is described a method of positioning a bone plate, comprising: attaching a grasping instrument to the plate to immobilize the grasping instrument relative to the plate such that an aperture in the grasping instrument aligns with a screwhole in the plate; immobilizing the grasping instrument relative to a structure immobilized on the bone to thereby immobilize the plate relative to the bone; and inserting a bone screw through the aperture in the grasping instrument and into the screwhole.

In another aspect, there is described a bone plate system comprising: a base plate having a first borehole and a second borehole both positioned adjacent a first edge of the plate, wherein the first borehole is positioned a first distance from the first edge and the second boreholes is positioned a second distance from the first edge and wherein the first distance differs from the second distance; the base plate also having a third borehole and a fourth borehole both positioned adjacent a second edge of the plate, wherein the third borehole is positioned a third distance from the second edge and the fourth boreholes is positioned a fourth distance from the second edge and wherein the third distance differs from the fourth distance.

In another aspect, there is described a bone plate system comprising: a base plate adapted to be attached to a bone, the base plate having a at least one open-ended slots for receipt of a bone screw, the slot positioned along a centerline of the plate.

In another aspect, there is described a bone plate system comprising: a base plate adapted to be attached to a bone, the base plate having two or more apertures for receipt of bone screws, the apertures positioned along a centerline of the plate.

In another aspect, there is described a method of placing a bone plate on a spine of a patient, comprising: a base plate adapted to be attached to a bone, the base plate having a pair of open-ended slots for receipt of bone screws the slots positioned along a centerline of the plate; placing the base plate on the spine such that the centerline is substantially aligned with a long axis of the spine; and securing the base plate to the spine.

In another aspect, there is described a bone plate system, comprising: a base plate having at least two apertures aligned along an axis; at least one enlarged region of the base plate extending laterally outward from the axis, wherein the enlarged region includes at least one borehole for receipt of a bone screw; and a central region of the base plate having a reduced lateral dimension relative to the enlarged region.

Other features and advantages should be apparent from the following description of various embodiments, which illustrate, by way of example, the principles of the disclosed devices and methods.

### BRIEF DESCRIPTION OF THE DRAWINGS

FIG. 1 shows a perspective view of a bone fixation plate with a plurality of bone screws positioned within mounting boreholes.

FIG. 1B shows a top down view of another embodiment of the plate 105.

FIG. 2A shows a perspective view of the first plate embodiment without the bone screws.

FIG. 2B shows a top view of a portion of the plate.

FIG. 2C shows a cross-sectional view of the plate along line B-B of FIG. 2B.

FIG. 3 shows another embodiment of the screw retainers on the plate.

FIG. 4 shows a partial transparent top view of the plate of FIG. 3.

FIG. 5 shows a cross-sectional view of the plate of FIG. 3 along line A-A.

FIG. 6 shows an instrument that is adapted to place and immobilize the plate prior to bone screw placement.

FIG. 7 shows an exploded view of the instrument.

FIG. 8 shows a partial transparent view of the actuation handle of the instrument.

FIG. 9 shows a plate positioned over a pair of vertebral bodies V1 and V2.

FIGS. 10 and 11A show the plate anchored to the vertebral bodies via the bone screws with the placement instrument removed.

FIGS. 11B and 11C illustrate an example of a plate used to fixate three vertebral bodies.

FIG. 12 shows exemplary basis measurements that can be used to select a plate size

FIG. 13 shows a plate with an exemplary plate length d3 that has been selected for use.

FIGS. 14-17 show the plate of a selected plate size positioned on the vertebral bodies.

FIG. 18A shows a perspective view of another bone plate embodiment.

FIG. 18B shows the plate of FIG. 18A in an exploded state.

FIGS. 18C and 18D show enlarged views of a portion of the plate in the region of openings for retaining clips.

FIG. 19 shows a top, cross-sectional view of the plate.

FIG. 20 shows a cross-sectional view of the plate with the retaining clip sections positioned over or within the boreholes.

FIG. 21 shows a perspective view of the plate with bone screws seated in the boreholes.

FIG. 22 shows a cross-sectional view of the plate showing the interaction between the retaining clips and the heads H of the bone screws.

FIG. 23A shows another embodiment of a plate that is similar to the plate shown in FIG. 18A.

FIG. 23B-23E shows another embodiment of a plate.

FIGS. 24-26 show a plate having another embodiment of a retainer member.

FIGS. 27-29 show alternative self-deploying retaining members that can be placed in the central aspect of a plate and used to retain both screws at each vertebral level.

FIG. 30 shows an alternate embodiment of a plate.

FIGS. 31A and 31B show perspective views of a modular distraction screw.

FIG. 31C shows an enlarged cross-sectional view of the interface between the distal portion segment and the proximal segment of the distraction screw.

FIGS. 32A-32C show various views of the distal segment of the distraction screw.

FIG. 33A shows a distraction screw placed into each of the vertebral bodies V1 and V2 above and below the disc space to be fused

FIG. 33B shows the distraction screws disassembled such that the proximal segments are detached from the distal segments.

FIG. 34 shows the attached distal segments with each head oriented perpendicular to the disc space S.

FIGS. 35 and 36 shows the plate after being lowered onto the distal segments.

FIG. 37 shows the plate secured to the bone using bone screws.

FIG. 38A shows an alternative embodiment of a plate.

FIG. 38B shows the plate of FIG. 38A in an exploded state.

FIG. 39 shows another embodiment of a plate.

FIG. 40 shows the plate of FIG. 39 attached to bone.

FIG. 41A-41B shows another embodiment of a plate.

FIG. 42A shows a perspective view of yet another embodiment of a plate.

FIG. 42B shows a cross-sectional view of the plate.

FIGS. 43A-D shows the plate of FIG. 42A with two screws positioned in slots of the plate.

FIGS. 44 and 45 show a plate formed of two segments.

FIG. 46 shows another embodiment of a plate comprised of an elongated structure having a single line of boreholes.

### DETAILED DESCRIPTION

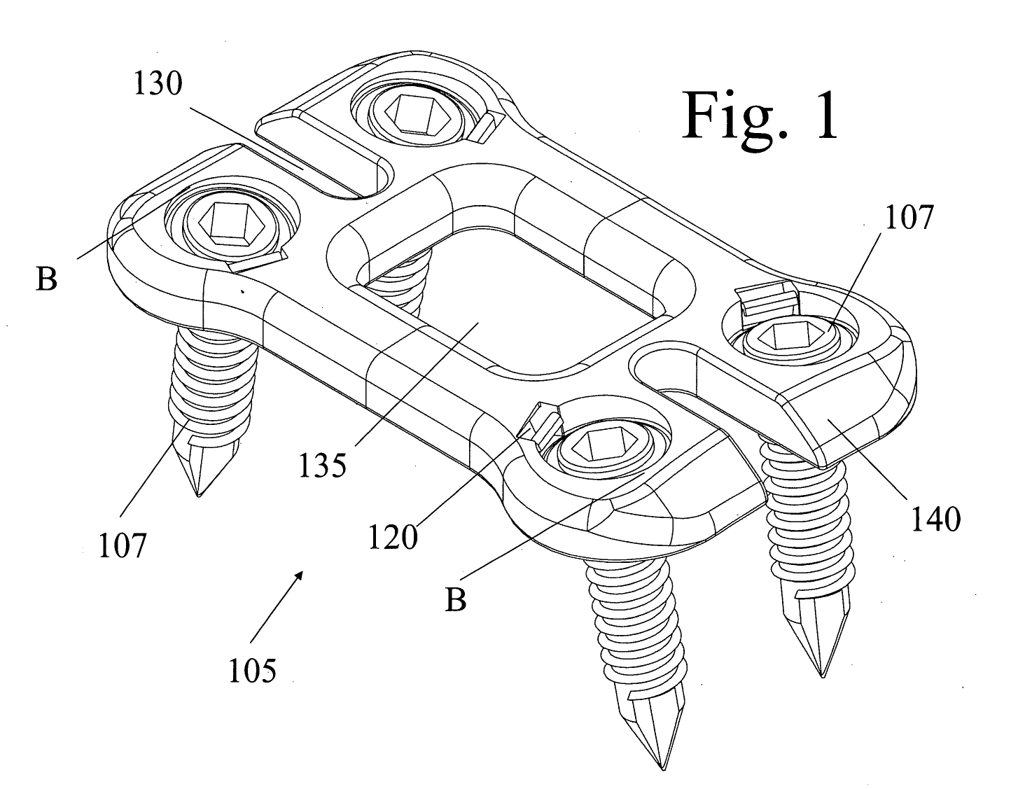

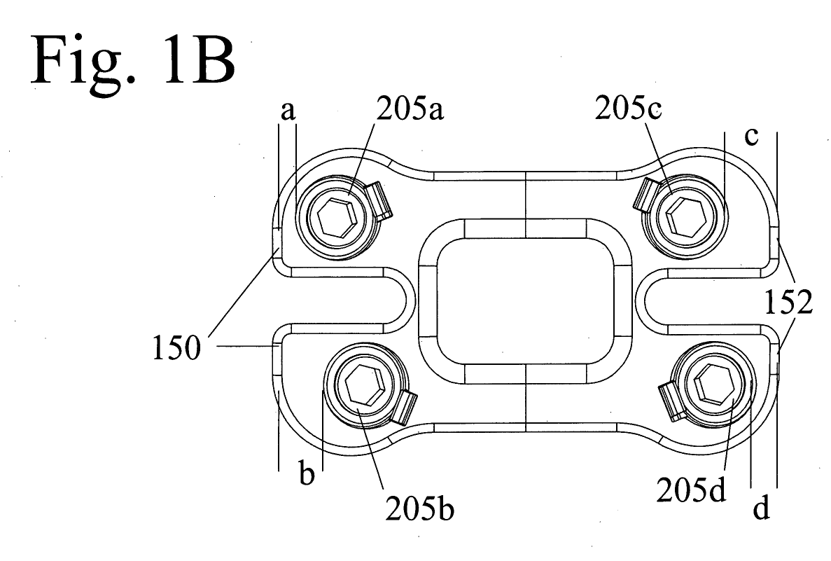

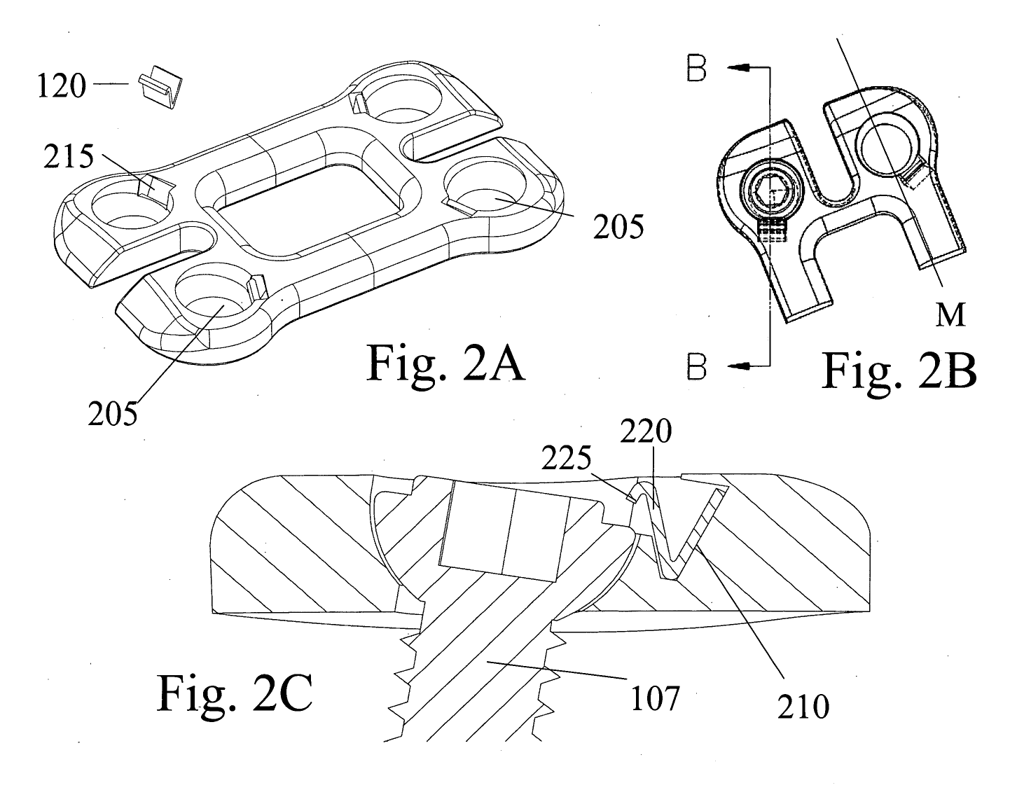

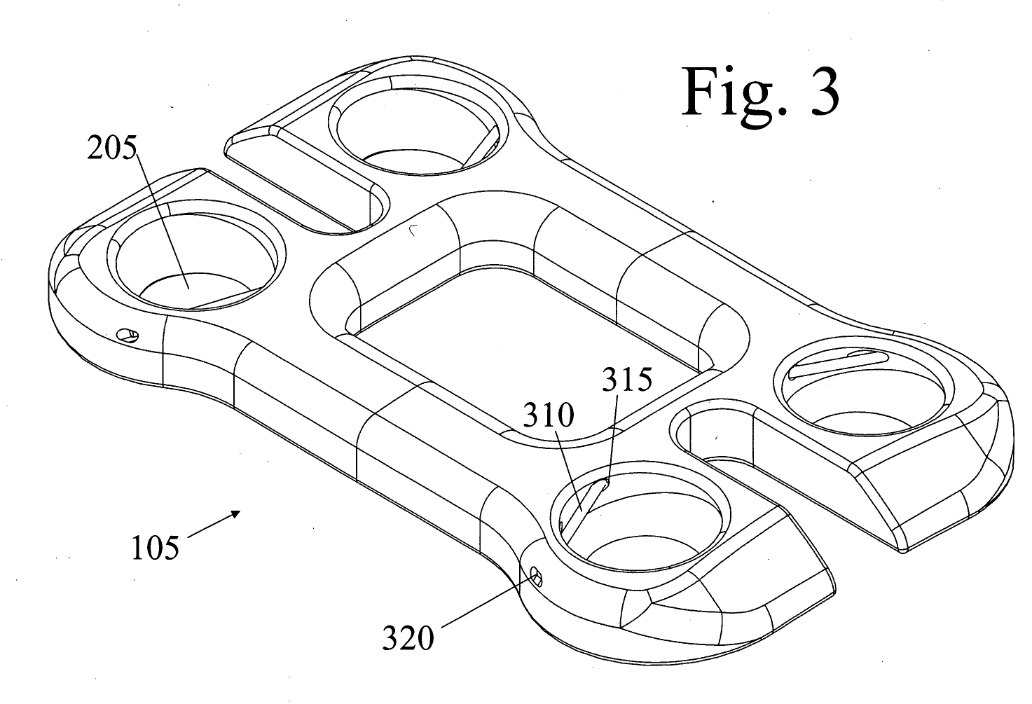

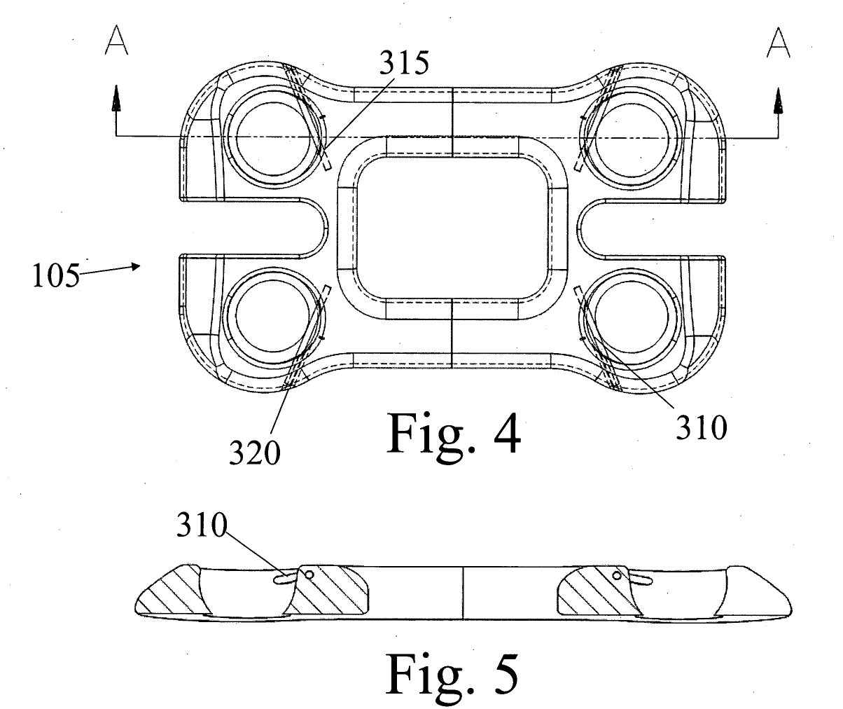

FIG. 1 shows a perspective view of a bone fixation plate 105 with a plurality of bone screws 107 positioned within mounting holes in the plate. FIG. 2A shows a perspective view of the plate 105 without the bone screws. FIG. 2B shows a top view of a portion of the plate 105 and FIG. 2C shows a cross-sectional view of the plate along line B-B of FIG. 2B.

With reference to FIGS. 1 and 2A-2C, the plate 105 includes one or more boreholes 205 (FIG. 2) that are each sized and shaped to receive at least one bone screw 107. In the embodiment of FIG. 1, a borehole 205 is positioned substantially adjacent to each of the four corners of the plate 105, although it should be appreciated that the position of each borehole on the plate 105 can vary. Each of the bone screws 107 generally comprises an elongated shank portion that extends downwardly from an enlarged screw head sized to fit inside the borehole 205. For each screw 107, the screw head is sized to fit through an upper end of the borehole 105. The head is larger than the bottom region of the borehole so that the totality of the head cannot be advanced beyond the bottom of the borehole. In this manner, as shown in FIG. 2C, the bone screw 107 can be positioned through the borehole 205 such that the shank extends through the bottom of the borehole but the head is retained within the borehole. As best shown in FIGS. 2A-2C, the boreholes 205 can include surfaces that are sized and shaped to compliment the outer surface of the head of the respective bone screw 107. Thus, the borehole surfaces can be rounded or concave to permit a correspondingly rounded head of the screw to rotate or move in one or more axis.

FIG. 1B shows a top down view of an embodiment of the plate 105. As mentioned, the plate 105 can include four boreholes 205 (or alternately elongated or slotted boreholes or open ended slots, or combinations thereof), which are referred to individually as boreholes 205a, 205b, 205c, and 205d. Each of the boreholes can be positioned at different distances relative to the adjacent edge of the plate. For example, the borehole 205a is a distance “a” from the edge 150 and the borehole 205b is a distance “b” from the edge 150. Likewise, the borehole 205c is a distance “c” from the edge 152 and the borehole 205d is a distance “d” from the edge 152. The distances a, b, c, and d can be measured from the plate edge to any constant reference point on each borehole, such as outer edge of the borehole or the center point of the borehole. In one embodiment, the distances a, b, c, and d are equal while, in other embodiments, the distances a, b, c, and d may be different. Alternately, some of the distances can the same while others are different according to various combinations. For example, distances a and d can be equal to one another while distances b and c are also equal to one another but different from distances a and d.

With reference again to FIG. 1, the plate 105 includes one or more screw retainers comprised of self-deploying retaining clips 120 that communicate with respective boreholes 205. The retaining clips 120 function to prevent the respective bone screws 107 from backing out of the boreholes 205 through the upper surface of the plate. The screw retainers described herein are self-deploying in that the screw retainers automatically disengage from the respective screw hole or borehole as the screw is inserted without requiring a surgeon to perform a separate step or manually move the retainer to the disengaged position. When disengaged, the screw retainer does not block the screw from entering or exiting the screw hole and when engaged, the screw retainer prevent the screw from being removed from the screw hole.

FIG. 2A shows a retaining clip 120 in an exploded state relative to the plate 105. The retaining clip 120 generally comprises a resilient structure having a surface 210 that is retained within cavity 215 on plate 105 adjacent to the borehole 205. The retaining clip 120 further includes a retainer portion 220 that is sized and shaped to resiliently engage the head of the bone screw 107 when positioned in the borehole 205. That is, the retainer portion 220 engages the head (or some other portion) of the bone screw 107 to prevent the bone screw from backing out of the borehole 205 after the bone screw has been positioned therein. The retaining clip or any embodiment of the retainer members can be sized such that it is positioned only along a portion of the circumference or perimeter of the respective borehole and does not entirely circle the perimeter of the borehole. In addition, the retaining clip is preferably positioned on the inferior and/or lateral aspect of the borehole with respect to the longitudinal midline M of the borehole.

The retaining clip 120 is configured such that the retainer portion 220 yields to the advancing head of the bone screw 207 as the screw is inserted through borehole 205 into the underlying bone. Thus, a surgeon is not required to separately move the retainer portion 220 out of the way when inserting the bone screw 107 through borehole 205. Once the head of the bone screw 107 has been seated within a seat in the borehole 205, the retainer portion 220 returns to its neutral position and overlies the screw head so as to prevent screw pull-out. Thus, the retaining clip 120 (as well as the other retainer members described herein) transitions between an engaged state wherein the retaining clip engages the screw head to prevent the screw from backing out of a plate, and a disengaged state wherein the retaining clip permits the screw to be inserted into the borehole. The retaining clip automatically transitions to the disengaged state as the screw is inserted into the and automatically transitions to the engaged state once the screw has been seated in the borehole.

In the embodiment of FIGS. 1-2C, the retainer portion 220 and the attachment portion 210 of the retaining clip 120 collectively form a “V” shaped structure that resiliently yields to the screw head during insertion. In a default state, the retainer portion 220 extends over at least a portion of the borehole to at least partially occlude the top of the borehole. As the bone screw 107 is inserted into the borehole 205, the screw head pushes the retainer portion 220 toward the perimeter of the borehole and permits the screw head to sit within the borehole. Once the screw head is seated, the retainer portion 220 snaps back into the default state to at least partially occlude the borehole and prevent backout of the screw. The retainer portion 220 includes a screw engagement structure 225 that extends toward the screw head. If it is desired to remove the bone screw 107 from the borehole 205, the retainer portion 220 can be reversibly deflected out of the way so that it no longer blocks the screw head during removal.

With reference again to FIG. 1, each end of the plate 105 has a central channel 130 that is adapted to receive a distraction screw. Each central channel 130 is an elongated channel that extends from one end of the plate toward a central region of the plate. The plate 105 further includes a central opening 135 that can permit x-ray evaluation of a bone graft after placement. The channel 130 preferably extends away from leading plate edge 140 and towards central opening 135 so that the channel length is greater than the distance from the leading edge 140 to the superior border B (FIG. 1) of borehole 205. In addition, the leading edge 140 of the plate 105 can be tapered to provide a gently sloping profile and minimize the possibility of impingement upon adjacent structures such as the esophagus. The latter lies immediately anterior to plates implanted in the cervical spine and food may be hindered as it travels down the esophagus by the shelve-like profile of a non-tapered leading edge.

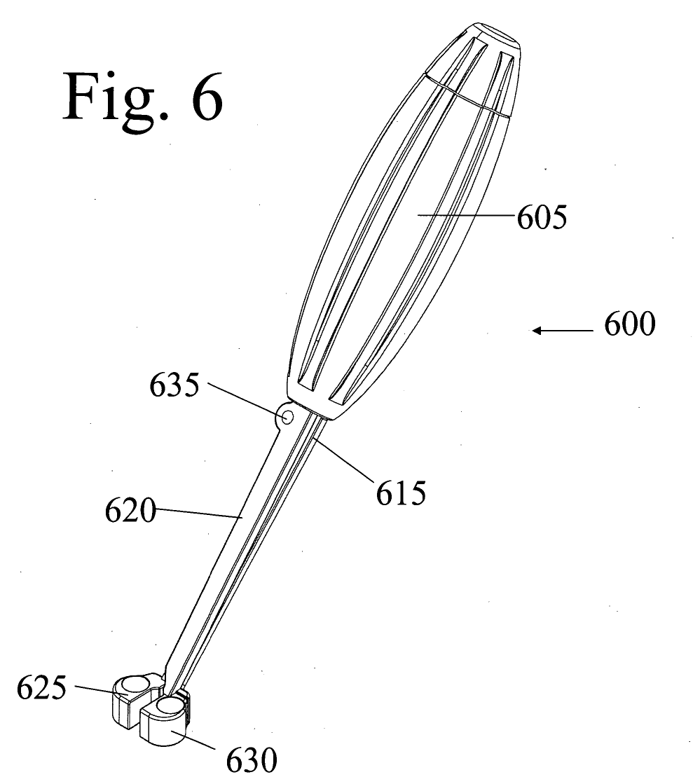

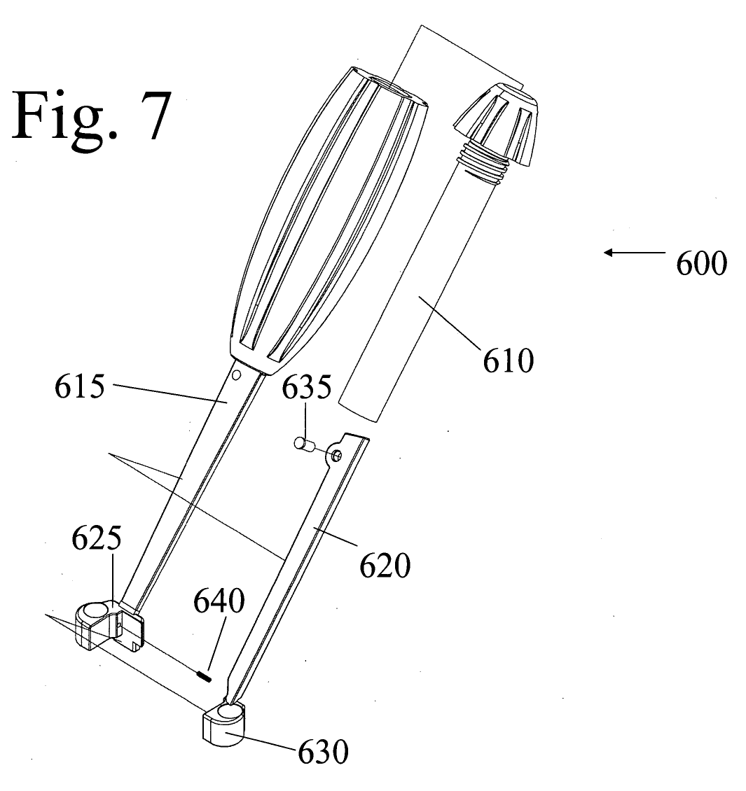

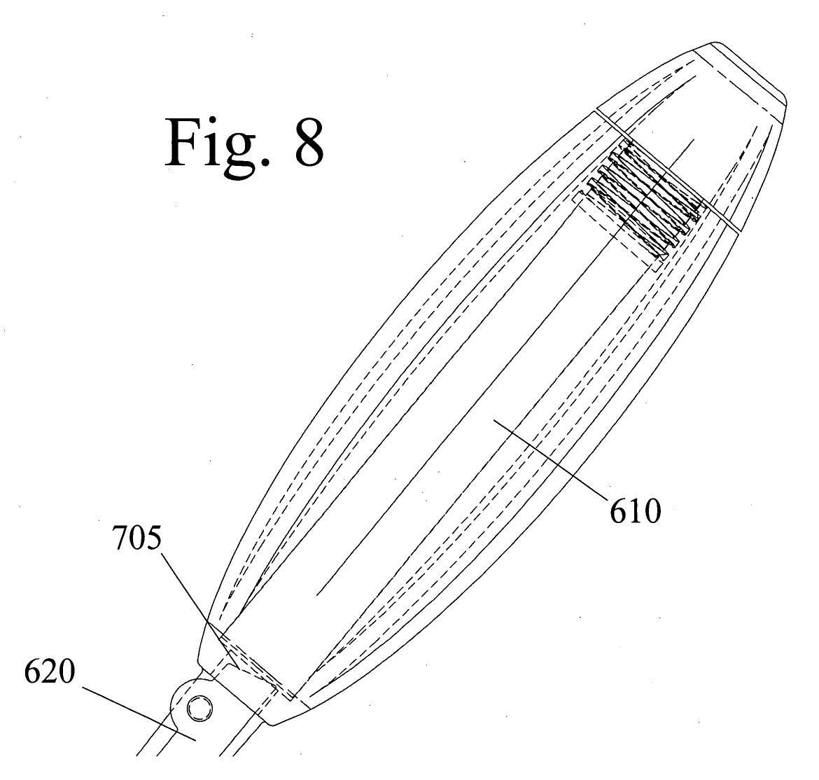

FIG. 3 shows another embodiment of the screw retainers on the plate 105. FIG. 4 shows a partial transparent top view of the plate with the screw retainer and FIG. 5 shows a cross-sectional view of the plate. In this embodiment, each of the screw retainers comprises a screw retaining member 310 that at least partially occludes a respective borehole 205. The screw retaining member 310 is attached at a first end to a coupling location 315 on the plate 105. The second end of the screw retaining member 310 is movably positioned within an opening 320 in the plate The second end of the screw retaining member 310 is sized such that it can move within the opening 320 thereby permitting the screw retaining member 310 to resiliently move out of the way of the advancing screw. That is, the screw retaining member 310 can move and/or rotate about a fixation point within bore 315. The opening 320 permits movement of retainer member 310 in, substantially, a single plane but limits the movement of member 310 in other planes.

The retainer member 310 automatically moves out of the way of the advancing bone screw head as the screw is inserted through borehole 205 and into the underlying bone. Once the head is seated within the borehole, the retaining member 310 returns to its neutral position and covers a portion of the screw head so as to prevent screw pull-out. While shown as immobile within the medial opening 315 and mobile within the lateral opening 320, it is understood that, alternatively, member 310 can be made immobile relative to the lateral opening and mobile within the medial opening.

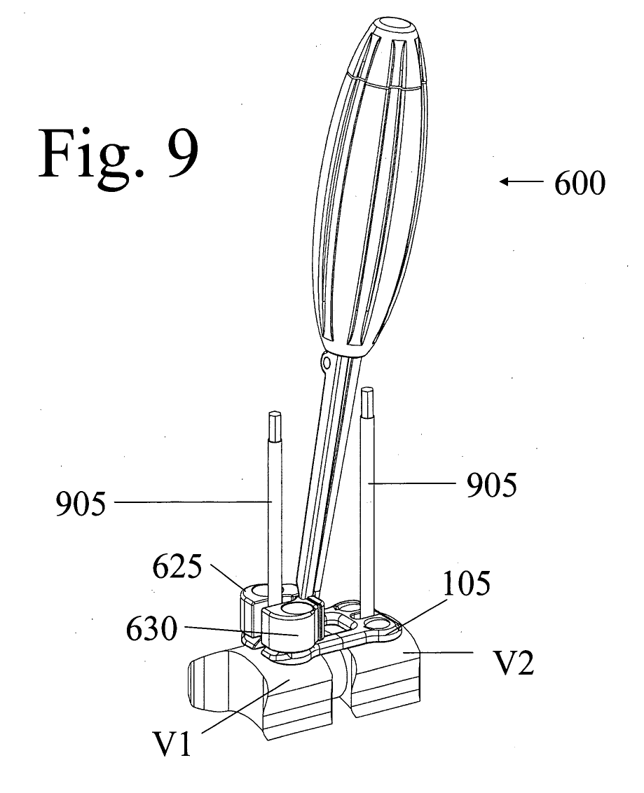

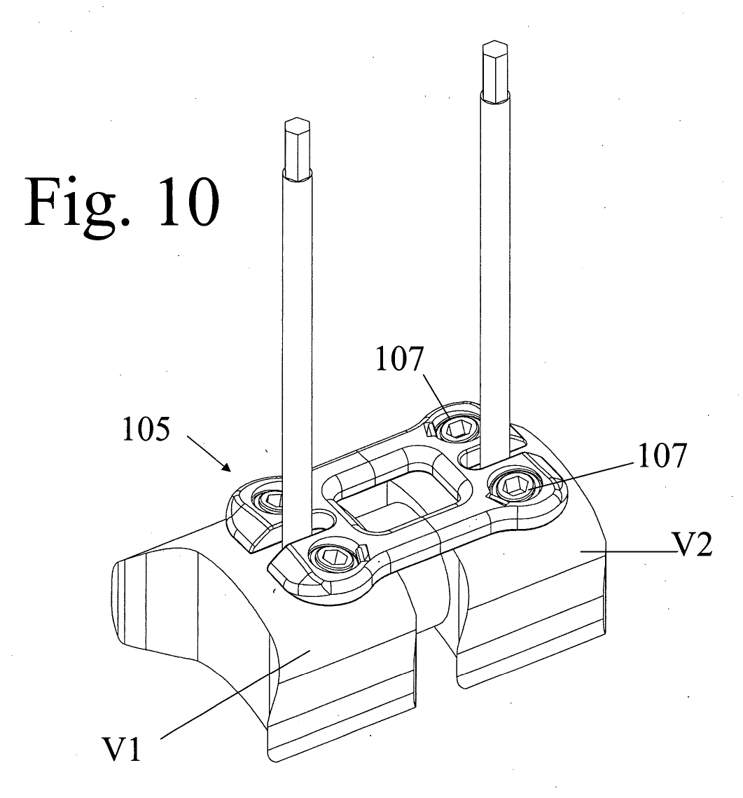

FIG. 6 shows an instrument 600 that is adapted to place into an operative field and immobilize the plate prior to the plate's bone screw placement. FIG. 7 shows an exploded view of the instrument 600. The instrument 600 includes an actuation handle 605 having a rotatably mounted internal member 610. Pair of holding arms 615 and 620 with respective graspers 625 and 630 extend downwardly from the actuation handle 605. The assembled instrument 600 functions similar to a pair of pliers. That is, the holding arms 615 and 620 are pivotably movable toward and away from one another about a pivot pin 635. A biasing member such as a spring 640 is interposed between the graspers 625 and 630 to bias the graspers 625 and 630 away from one another.

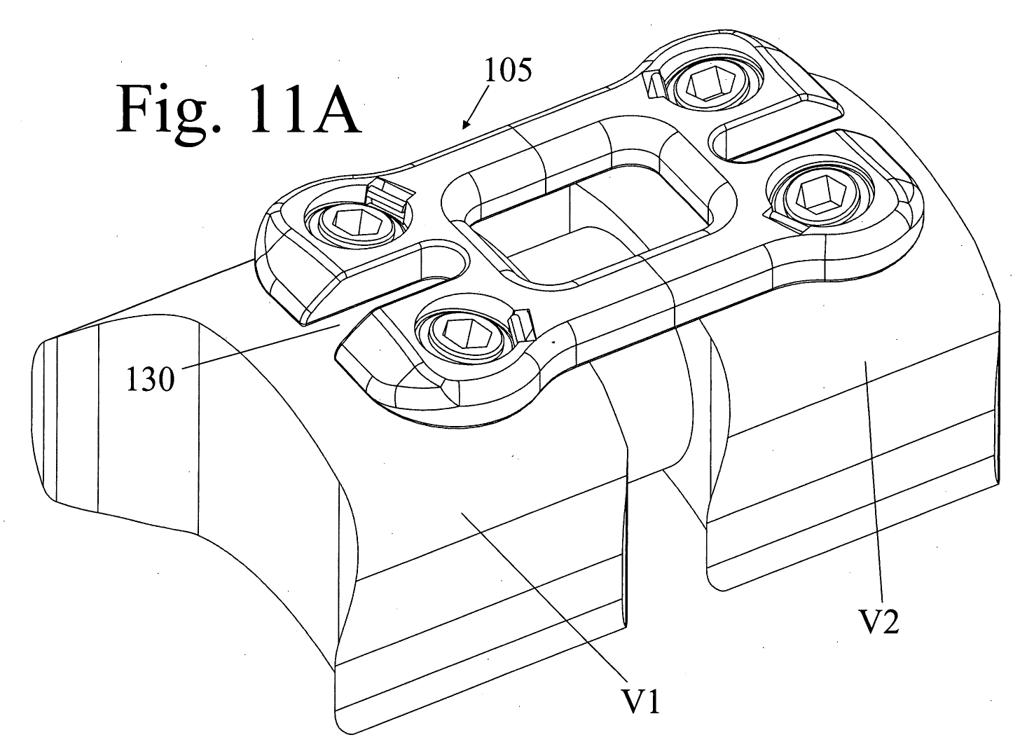

FIG. 8 shows a partial transparent view of the actuation handle 605. The internal member 610 is rotatably positioned inside the handle 605. A bottom edge of the internal member 610 abuts a sloped surface 705 on an upper edge of the holding arm 620. The internal member 610 can be rotated relative to the actuation handle 610 to cause the internal member to move along the axis of the handle 610 and push against the sloped surface 705 of the holding arm 620. This causes the holding arm 620 to pivot toward or away relative to the holding arm 615 depending on the direction in which the internal member 610 is moved. The bores within graspers 625 and 630 align with the screwholes in the plate when the graspers are attached to the plate. The bores within the graspers are sufficiently sized so as to permit passage of the bone screw (including head) through them and into the underlying plate boreholes. Further, the bores are also adapted to guide the drill that forms the bone holes prior to bone screw placement. The bores of instrument 600 may be adapted to guide the drill into a pre-determined, stationary trajectory or onto a variable angle trajectory.

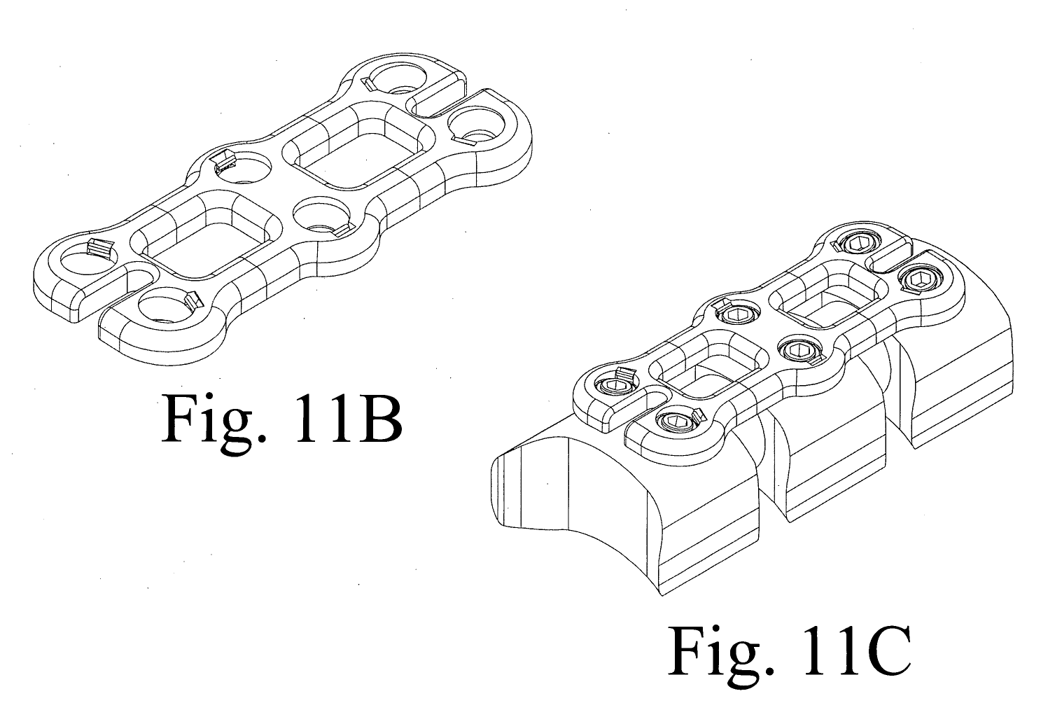

FIG. 9 shows the plate 105 positioned over a pair of vertebral bodies V1 and V2. A pair of distraction screws 905 are attached to the vertebral bodies V1 and V2. The plate 105 is positioned over the distraction screws 905 with the distraction screws 905 extending through the central channels 130 (FIG. 1) of the plate 105. The plate placement instrument 600 is attached to plate 105. The elongated channels 130 permit relative movement between the plate 105 and the distraction screws 905 along the long axis of the spine, which is aligned with the long axis of the channels 130. The relative position of the distraction screws 905 and the channels 130 limit horizontal plate movement and plate rotation relative to the underlying bone. When the plate 105 has been appropriately positioned in the vertical plane (i.e., along the long axis of the spine), instrument 600 is actuated so that graspers 625 and 630 close around and retain the distraction screw. That is, as the actuation handle is rotated and tightened, distraction screw 905 is wedged between mobile grasper 630 and stationary grasper 625. In this way, instrument 600 and the attached plate 105 are immobilized relative to the underlying bone.

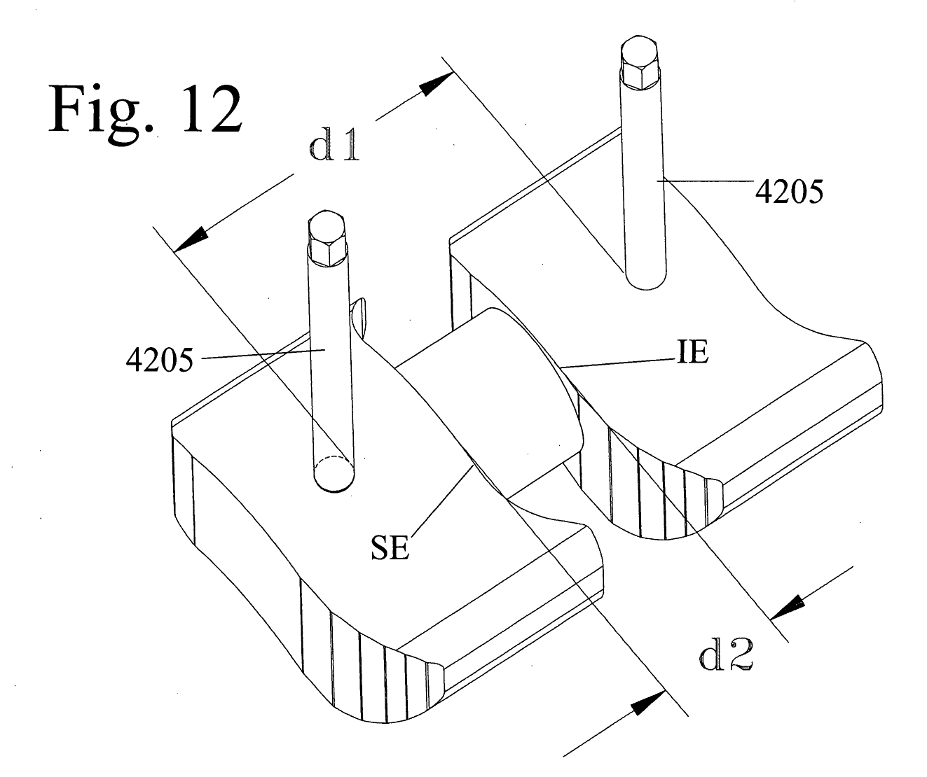

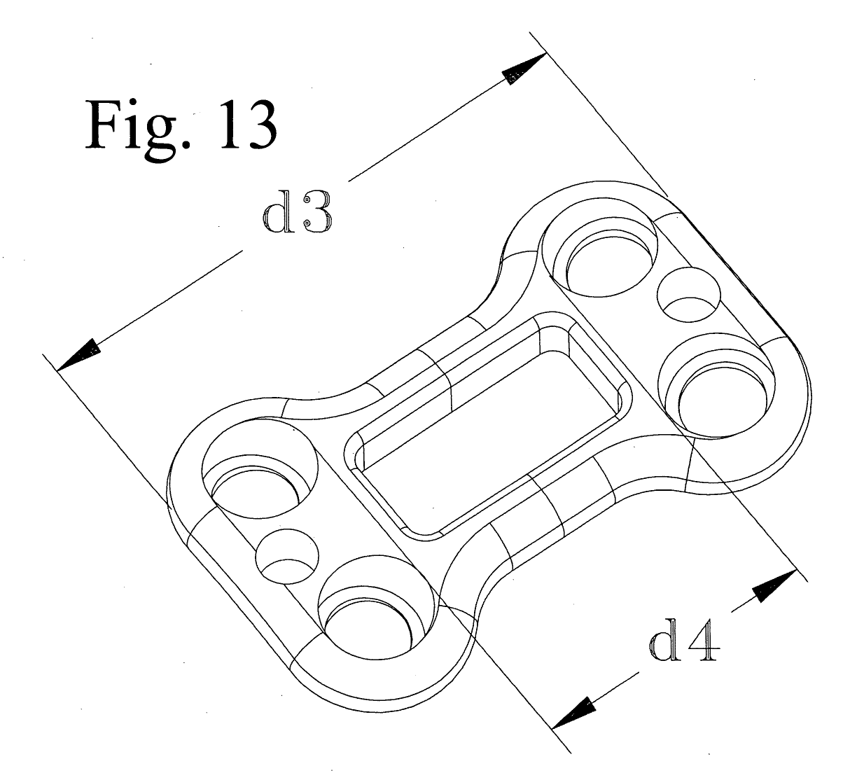

With the plate 105 immobilized relative to the distraction screws, the bores within graspers 625 and 630 of instrument 600 are used as a drill guide and, subsequently, as a conduit for bone screw placement. A pair of bone screws 107 are inserted through boreholes 205 of the plate 105 and into the underlying vertebral body V1. A shank portion of the bone screws can be screwed into the bone such that the head portion of the screw engages the plate and immobilizes the plate relative to the bone. The instrument 600 is then disengaged from the plate 105 and the distraction screw. Bone screws are placed through the vacant boreholes of plate 105 and into vertebral body V2. FIG. 10 shows the plate 105 anchored to the vertebral bodies via the bone screws 107 with instrument 600 removed. The distraction screws 905 are then removed thereby leaving the plate 105 attached to the vertebral bodies V1 and V2, as shown in FIG. 11A. (Alternatively, the distraction screws may be removed before placement of the bone screws into vertebral body V2.) The empty central channels 130 allow placement of distraction screws into the underlying bone at a subsequent operation without plate removal. This permits future extension of the fusion to an adjacent level or placement of an artificial disc at that adjacent level.

While the preceding embodiment fixates two adjacent vertebral bodies, plates that are used to fixate three or more bones can be similarly made by the sequential addition of additional bore hole. FIGS. 11B and 11C illustrate an example of a plate used to fixate three vertebral bodies.

Bone plates are manufactured and provided to the surgeon in a range of sizes that vary by a fixed amount. At the time of surgery, the surgeon must choose the plate that best fits the individual patient. Appropriate plate selection can be critical since a short plate may provide inadequate fixation and increase the likelihood of construct failure while a long plate may overly the adjacent, un-diseased disc spaces and unnecessarily restrict spinal mobility. Unfortunately, there is no current method that maximizes the likelihood of proper plate selection.

Once selected, the plate is positioned over the cervical spine and bone screws are used to attach it to the vertebral bodies. Since the bone screws are designed to be placed into the underlying bone at an angle, the bone holes are created by positioning the drill or self-drilling screws at an angle relative to the bone surface. Because of this, extensive “travel” of the plate and screws often occurs while the bone screws are being placed and, consequently, the plate may be poorly positioned at the surgical site. Some plating systems use a small pin fixator to temporarily immobilize the plate. This feature tries to minimize the extent of plate travel and expedite the plating procedure.

While intuitively appealing, use of temporary fixation pins is of little practical value. The screws used to distract the vertebral bodies during the bone work that precedes plate placement leave empty holes in the underlying bone. Since the distraction screws are larger than the pin fixators, the bone holes they leave behind will interfere with placement of the pin fixators. Further, attempts at pin placement away from the empty screw holes may lead to off-center and crooked plate placement.

Correct placement of the plate in the vertical plane is especially important to the maintenance of optimal bony alignment. With normal bone subsidence, the fixation plate permits movement along its own long axis. Thus, when the vertical axis of the plate and that of the spine are not properly aligned, the plate will further worsen the bony alignment as the vertebral bones subside.

There is now described a method for selection of a plate length. After completion of the discectomy and placement of the bone graft, the distance between the inferior edge of the upper vertebra and the superior edge of the lower vertebra is measured. That distance forms a basis measurement, which is used to select the plate. In one embodiment, the plate is chosen so that the plate length exceeds the distance between the vertebral edges by a fixed amount. Alternatively, the distance between the distraction screws may be used as another basis measurement. Since the distances between all points on the plate are known, the plate selection may be based on the difference in distance between the chosen basis measurement and another set of fixed plate points.

Once selected, the plate is immobilized using temporary fixation pins. These pins can have a diameter equal to or greater than the diameter of the distraction screws used to perform the discectomy. This insures that the temporary pin will be capable of adequately immobilizing the plate and significantly reduces the likelihood of improper plate placement.

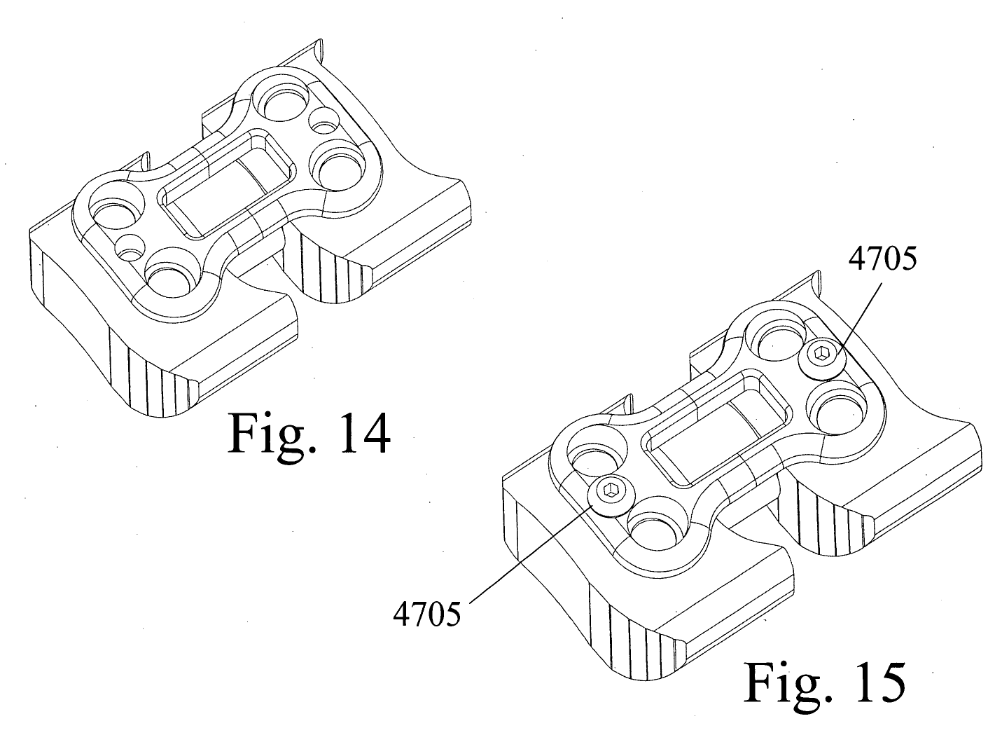

FIG. 12 shows exemplary basis measurements that can be used to select a plate size. In accordance with a method of selecting plate size, a basis measurement is selected wherein the basis measurement is used as the basis for selecting the plate size. In one embodiment, the distance d1 is measured and used as the basis measurement wherein d1 is the distance between the two distraction screws 4205. In another embodiment, the distance d2 is measured and used as the basis measurement wherein d2 is the distance between the inferior edge IE of the upper vertebra and the superior edge SE of the lower vertebra. In another embodiment, the distance equal to the graft height may be measured and used as the basis measurement instead of distance d2. However, since the graft may be resting on uneven vertebral surfaces, use of the distance d2 will provide a more accurate determinate of plate length than the graft height.

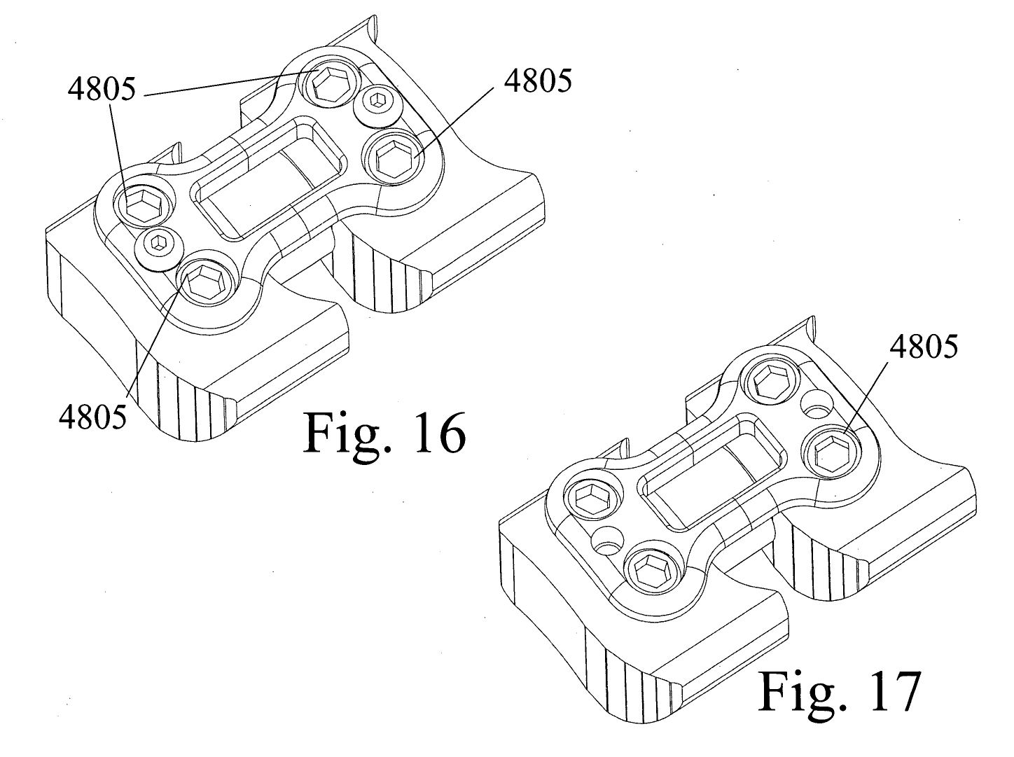

FIG. 13 shows a plate with an exemplary plate length d3 that has been selected for use. The plate length d3 differs from the basis measurement (e.g., d1 or d2) by a specified amount. The specified amount is a constant and depends on which basis measurement is used. Use of d2 as the basis measurement, for example, lead to selection of a plate length d3 equal to d2 plus constant k2. Constant k2 is specific to basis measurement d2. Use of the basis measurement d1 would require a different constant k2 than if d2 is used. In order to further tailor the plate to the size of the individual patient, the constant k2 for each basis measurement may also depend on the height of the patient's vertebral bodies. That is, a tall patient with tall vertebral bodies would have a larger constant added to the basis measurement and thereby receive a longer plate than would a shorter patient with shorter vertebral bodies. Thus, a different constant can be used for tall vertebral bodies (i.e., tall patient) than for short ones.

It is understood that all points on any particular plate have a fixed and known relationship to one another. Thus, plate selection may be alternatively based upon the distance between other fixed plate points and the basis measurement. For example, the plate may be alternatively selected so that the distance d4 between the bottom edge of the upper screws and the top edge of the lower screws differs from the basis measurement by a fixed amount.

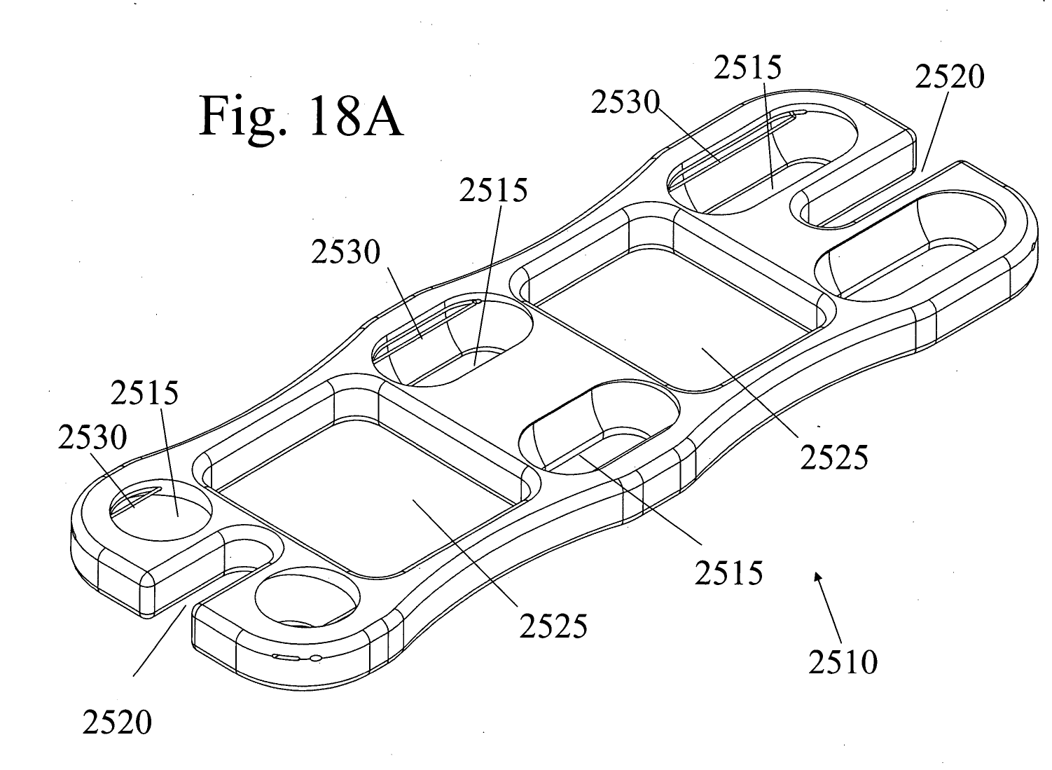

Once the plate size has been selected, a plate of the selected size is positioned onto the anterior aspect of the cervical spine as shown in FIG. 14. If desired, one or more pin fixators 4705 are used to immobilize the plate, as shown in FIG. 15. The pin fixators have shaft portions that are inserted into the bone. One or more bone screws 4805 are then placed into the underlying bone, as shown in FIG. 16. Preferably, the shaft portion of each fixator pin has a diameter equal to or greater than the shaft of the distraction screws. Finally, the fixator pins are preferably, but not necessarily, removed at the end of the plating procedure, such as shown in FIG. 17.

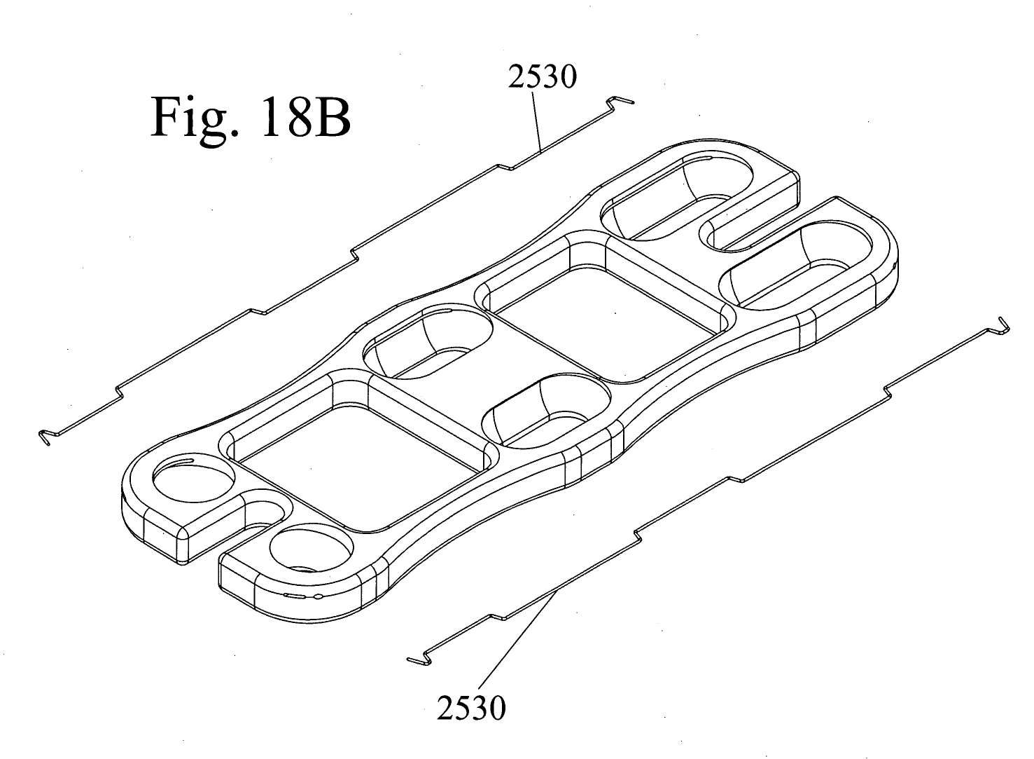

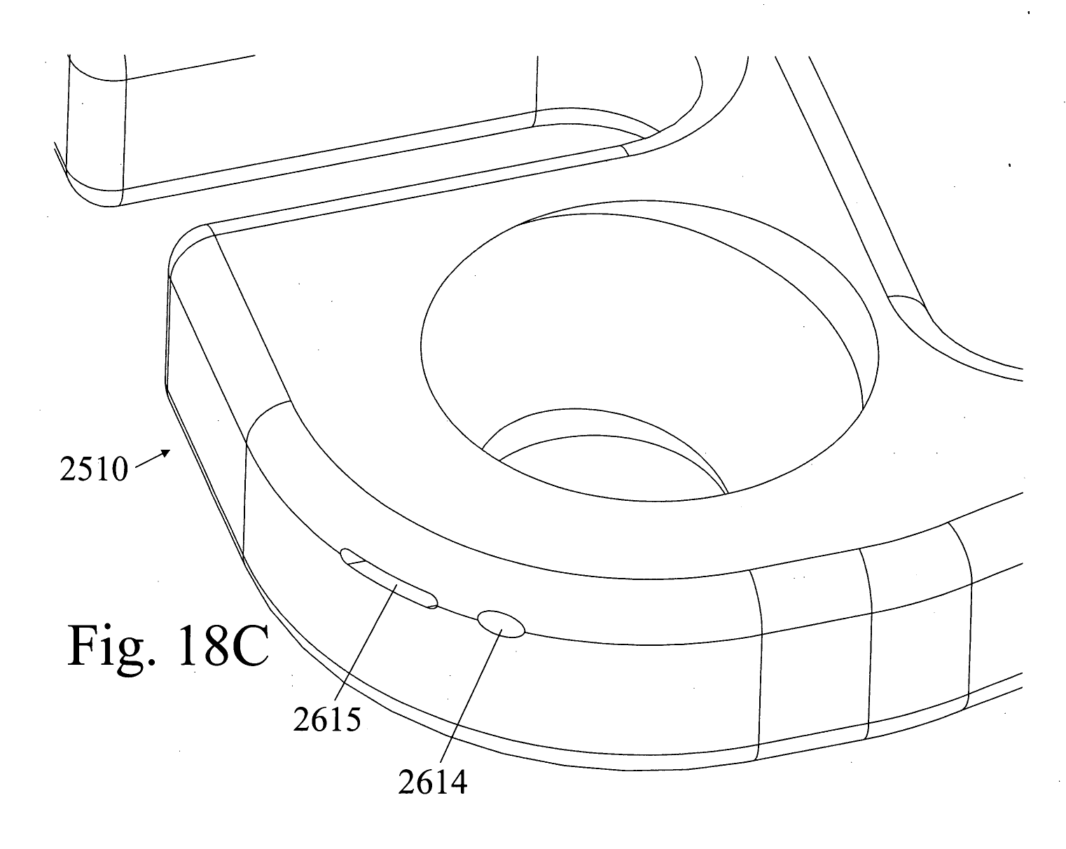

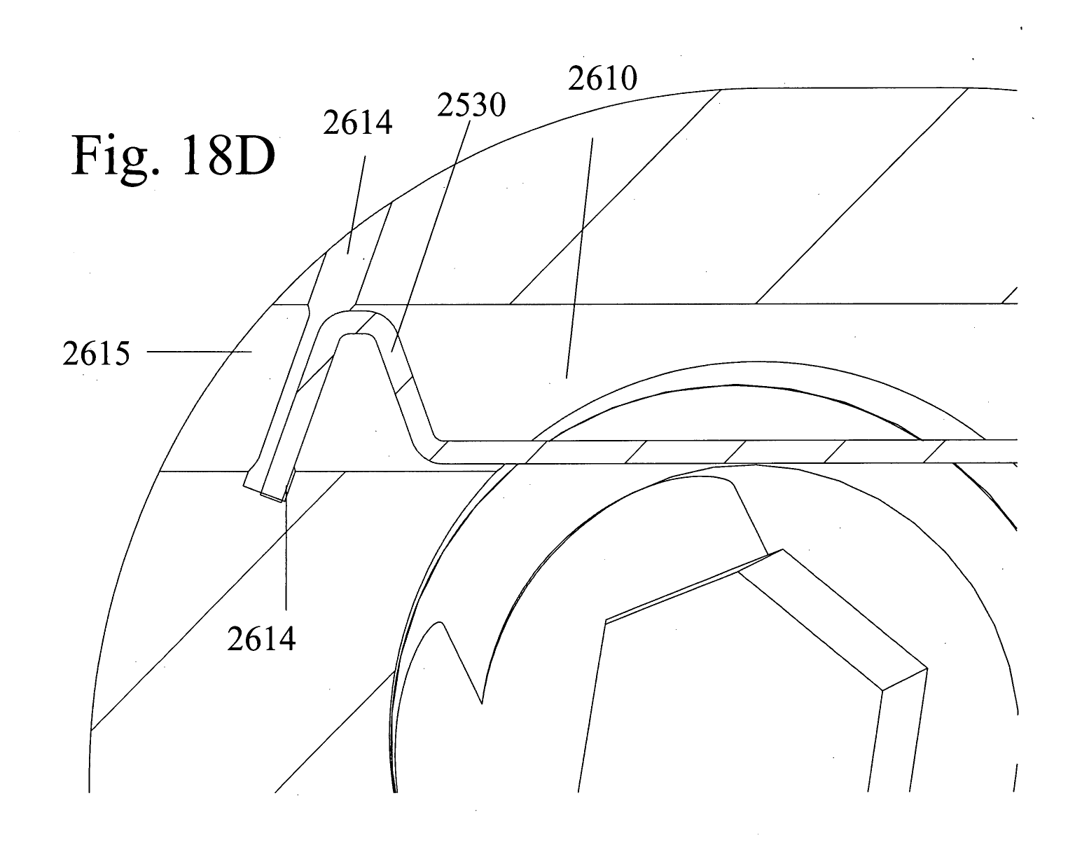

FIG. 18A shows a perspective view of yet another embodiment of a plate 2510. FIG. 18B shows the plate of FIG. 18A in an exploded state. The bone plate 2510 contains elongated or circular boreholes 2515 through which bone screw or similar fasteners pass into the underlying bone. The plate can be curved convexly in both the horizontal and vertical planes in order to conform to the anterior aspect of the cervical spine. When used in other spinal regions, the plate may be appropriately contoured to conform to the local anatomy.

The plate 2510 further includes a pair of elongated channels 2520 along the midline of the plate to aid with plate alignment and placement. Each of the elongated channels 2520 extend from an end of the plate toward the interior of the plate along a predetermined distance. In addition, the plate 2510 can include one or more central openings 2525 that can permit x-ray evaluation of a bone graft after placement.

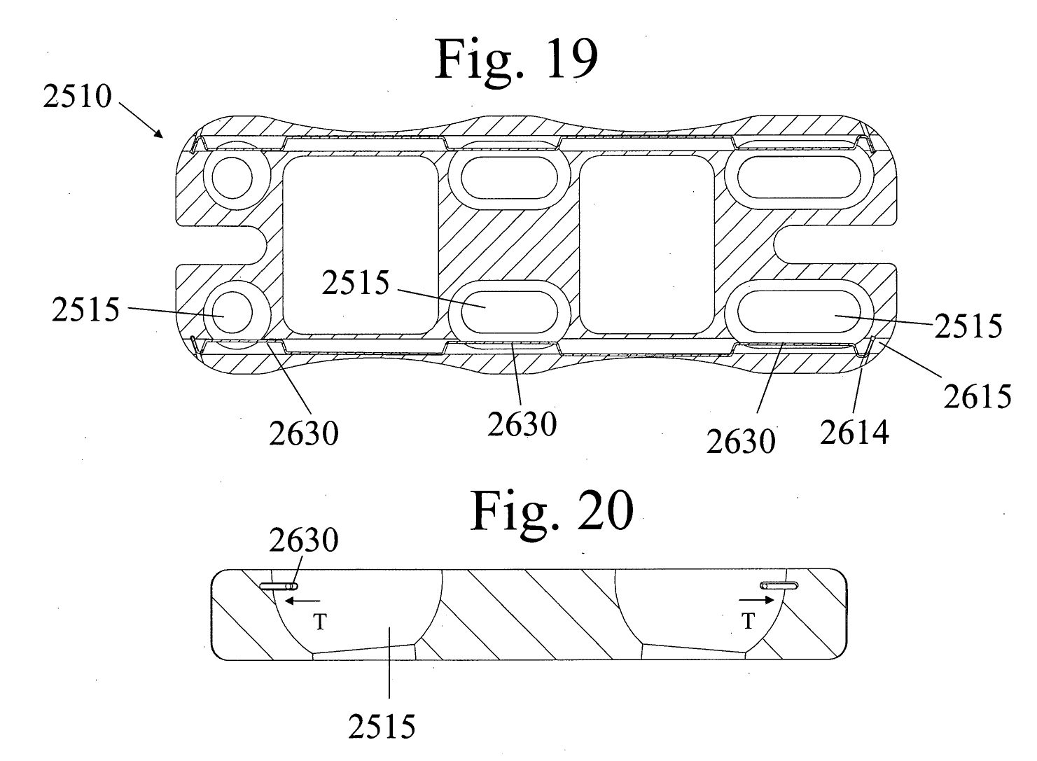

With reference to FIGS. 18B and 19, the plate includes one or more screw retainers comprised of elongated and resilient members 2530 that communicate with respective boreholes 2515. The retaining members 2530 function to prevent the respective bone screws in the boreholes 2515 from backing out through the upper surface of the plate. Each of the retaining members 2530 is an elongated structure positioned in a respective internal bore 2610 that extends through lateral sides of the plate 2510 along the direction of the longitudinal axis. The bores 2610 extend entirely through the plate 2510 such that openings 2615 (FIGS. 18C and 19) are located on the ends of the plate 2510. The openings 2615 provide ports through which the retaining members 2530 can be inserted into the bores 2610.

FIG. 18C shows an enlarged view of a portion of the plate 2510 in the region of one of the openings 2615. With reference to FIGS. 18C and 19, the opening 2615 is formed by the outer communication of the bore 2610. An additional opening 2612 is formed by a cross-drilled bore 2614 that functions to capture a portion of the retaining member 2530. As shown in FIG. 18D, an edge of member 2530 sits within the bore 2614 for fixation of member 2530 within the bore 2610. With advancement of the retaining member 2530 into the bore 2610, the edge of member 2530 snaps into the blind end of the bore 2614. In this way, member 2530 is held in place after positioning. The internal bore 2610 may be alternatively open on one end and closed on the other. In that configuration, the retaining member 2530 is captured into the bore 2614 on the open side alone.

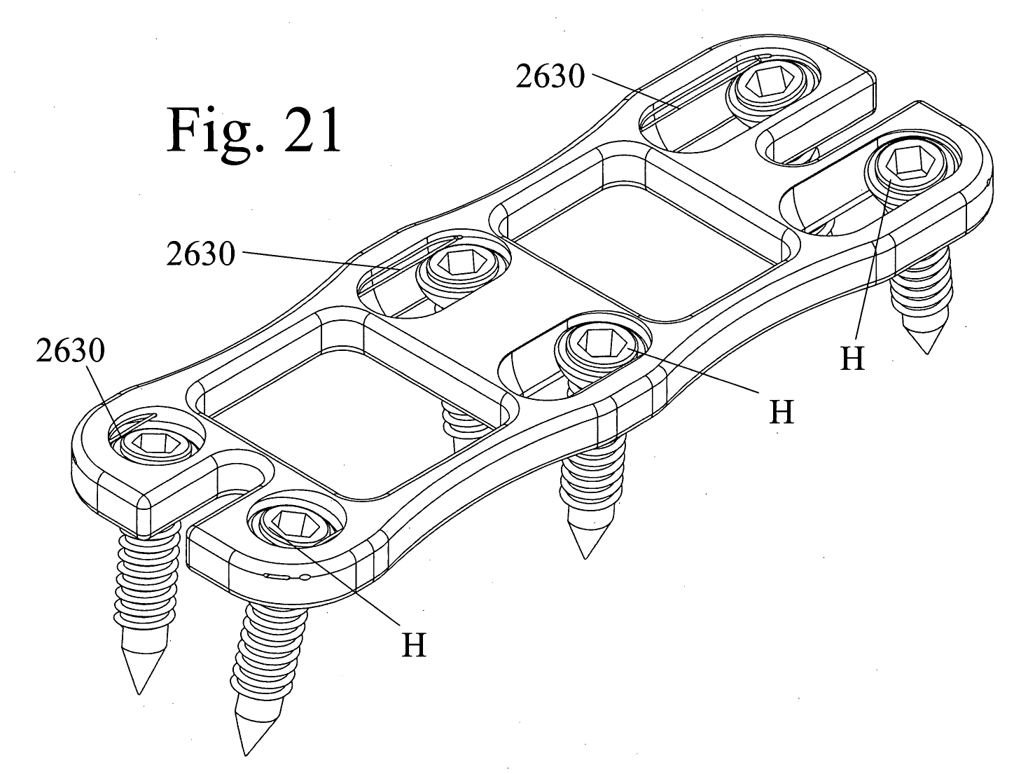

As shown in FIG. 19, each of the retaining members 2530 includes sections 2630 that extend at least partially over the boreholes 2515. FIG. 20 shows a cross-sectional view of the plate with the retaining member sections 2630 positioned over or within the boreholes 2515. The sections 2630 are resiliently positioned such that they can be pushed out of the way of the boreholes in response to insertion of a bone screw through the borehole and into the underlying bone. The retainer springs back into position once the bone screw has been seated in the borehole. Thus, the sections 2630 can be pushed away from interference with the borehole 2515 (as represented by the arrow T in FIG. 20) as the bone screw is inserted into the borehole. The sections 2630 then spring back in the opposite direction to the position shown in FIG. 20 once the bone screw has been seated in the borehole.

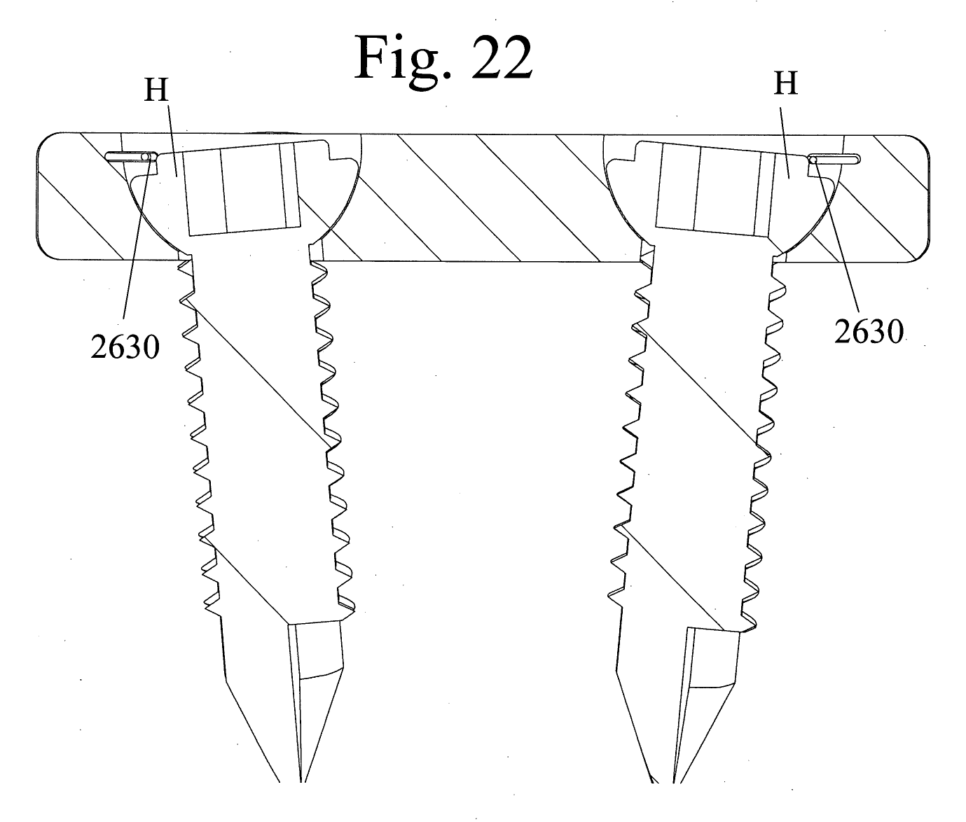

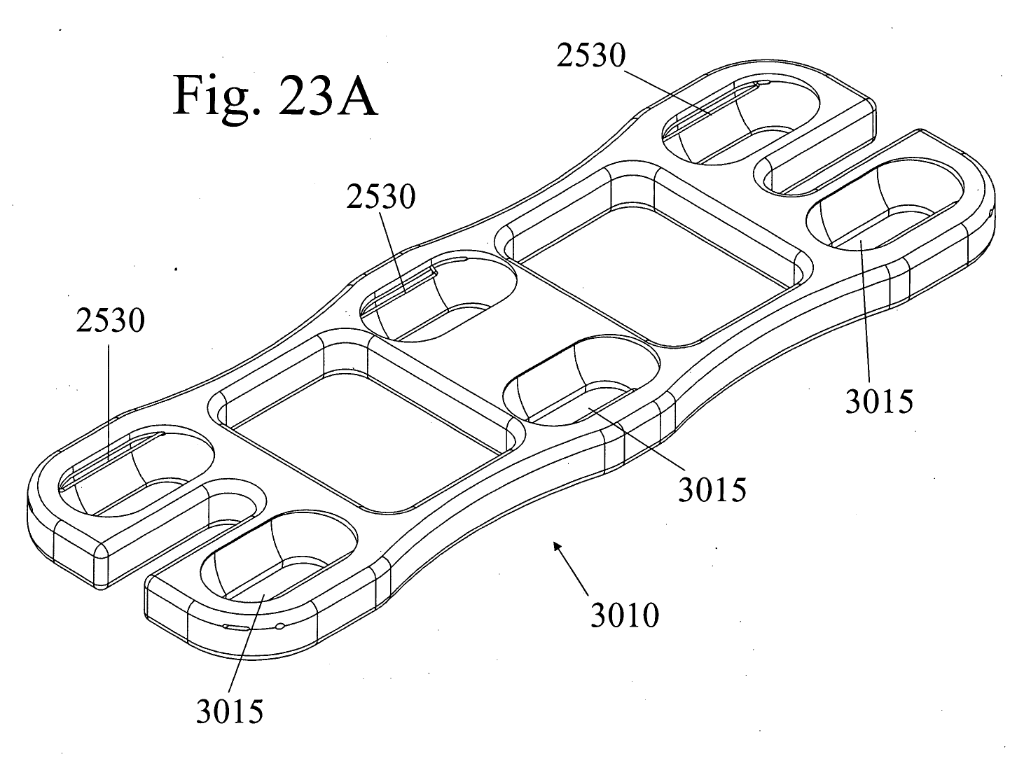

FIG. 21 shows a perspective view of the plate 2510 with bone screws seated in the boreholes 2515. At each borehole, section 2630 of retaining member 2530 covers a portion of screw head H and thus retains the bone screw within the borehole. FIG. 22 shows a cross-sectional view of the plate 2510 showing the interaction between section 2630 of the retaining members 2530 and the heads H of the bone screws. Sections 2630 are positioned so as to cover a portion of the heads H, such as at stepped surfaces on the heads H or some other region of the heads, and prevent screw back-out. If screw removal is desired, section 2630 can be displaced away from the midline so that it no longer overly the screw head. After release, the resilient retaining member will move back to re-cover the lateral portion of each borehole.

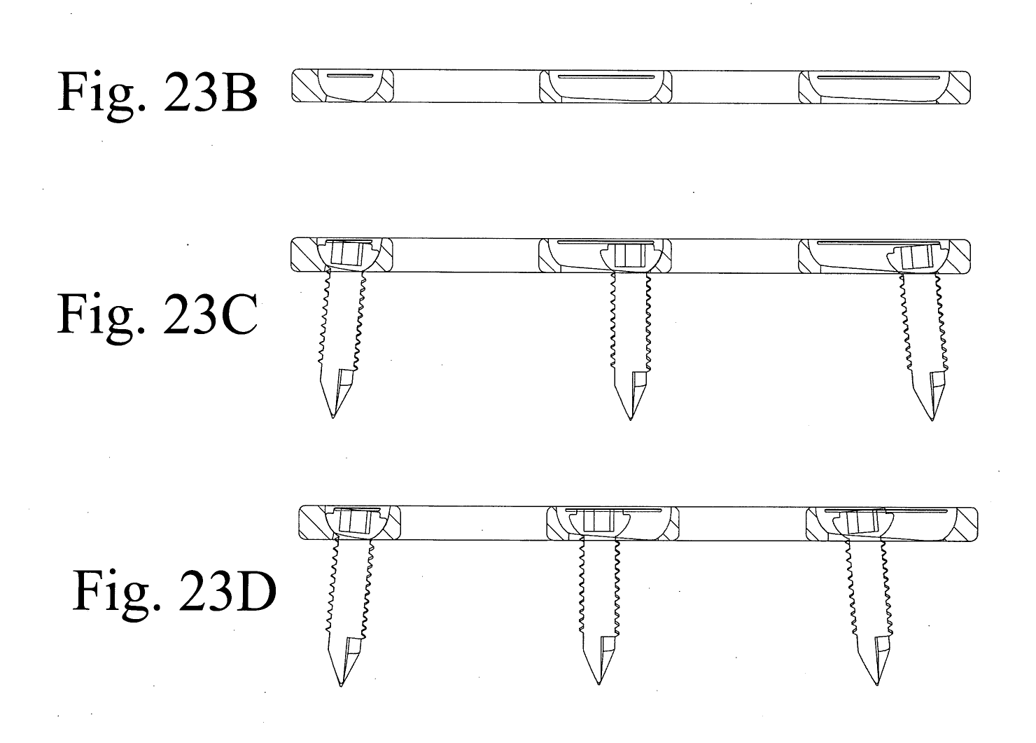

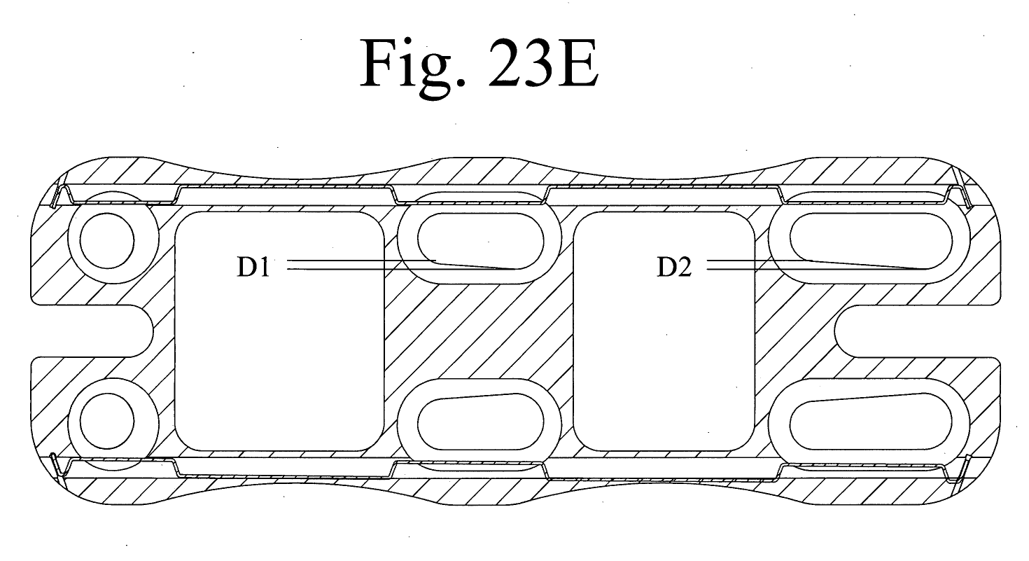

FIG. 23A shows another embodiment of a plate 3010 that is similar to the plate shown in FIG. 18A. However, the plate 3010 includes slotted boreholes 3015 at all positions. The plate 3010 includes a retainer member 2530 that is configured according to the retainer members described above with reference to FIGS. 18A22. FIG. 23B illustrates a cross-sectional view of another embodiment. In this version, the floor of one or more of the slotted boreholes are angled so that the distance between the floor of the borehole and the surface of the plate that abuts the bone increases as the slotted bore hole is transversed from its top (i.e., the region furthest from the non-slotted borehole) to its bottom (i.e., the region closest to the non-slotted borehole). FIGS. 23C and 23D show the plate with the screws attached. In FIG. 23C, the screws are shown immediately after insertion while FIG. 23D illustrates the plate and screws after screw and bone translation relative to the plate. Since the floor of each of the slotted boreholes is angled, the plate will necessarily wedge between the screw heads and the bone with progressive translation. In this way, translation produces greater resistance to further translation. A variable resistance to translation can be alternatively accomplished by the embodiment shown in FIG. 23E. In this version, the slotted boreholes progressively narrow as the slots are transversed from top to bottom. This feature may be produced by angling the medial wall of one or more boreholes laterally (as shown), angling the lateral wall medially or both.

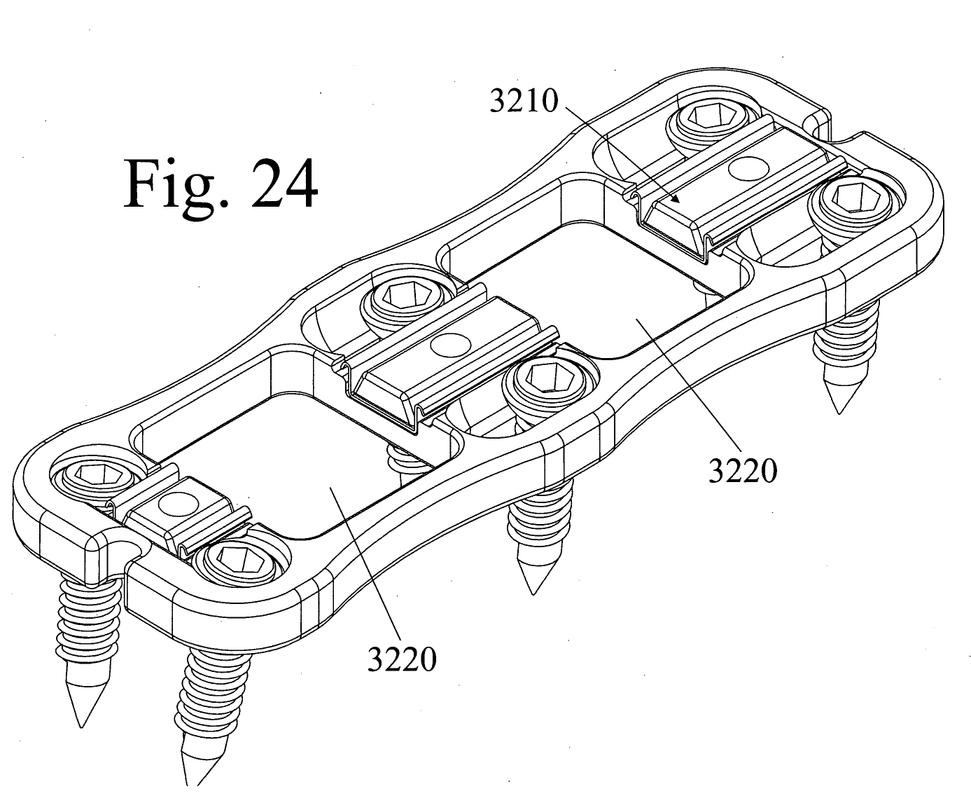

FIGS. 24-26 show a plate having another embodiment of a retainer member. In this embodiment, the retainer member comprises a clip 3210 that is positioned on the plate in communication with a pair of screws. The clip 3210 simultaneously engages a screw head on each side of the plate's midline so as to retain each screw within its borehole. As shown in the exploded view of FIG. 25, each clip 3210 includes an engagement member 3310 and a pair of locking members 3315 and 3320 that lock together to anchor member 3310 to the plate. As in the previous embodiments of the retainer member, the clip 3210 engages the screw head automatically as the screw is advanced into a borehole. The mechanism is also appropriately sized so that a central window 3220 can be retained.

With reference to the cross-sectional view of FIG. 26, the engagement member 3310 is a U-shaped structure that sits within an appropriately-sized seat 3410 such that edges of the engagement member 3310 engage the screw heads H. In this manner, the engagement members 3310 prevent the screw heads H from backing out of the borehole. The engagement member 3310 includes arms 3420 that can be automatically and resiliently pushed out of place by the screw head H as the screw is inserted through the borehole into the underlying bone.

As mentioned, the engagement member 3310 is secured to the plate using the locking members 3315 and 3320. The locking member 3315 is a cap that sits on top of the engagement member 3310. The locking member 3320 is a rivet-like structure that sits below the plate. A pin portion 3450 of the locking member 3320 fits through a hole in the plate and in the engagement member 3310 to lockingly fit within the cap of locking member 3315. In this manner, the engagement member 3310 is sandwiched between the locking member 3315 and the locking member 3320. The locking member 3320 has an enlarged head 3425 that is expanded so that the locking member does not fall out of engagement with the locking member 3315.

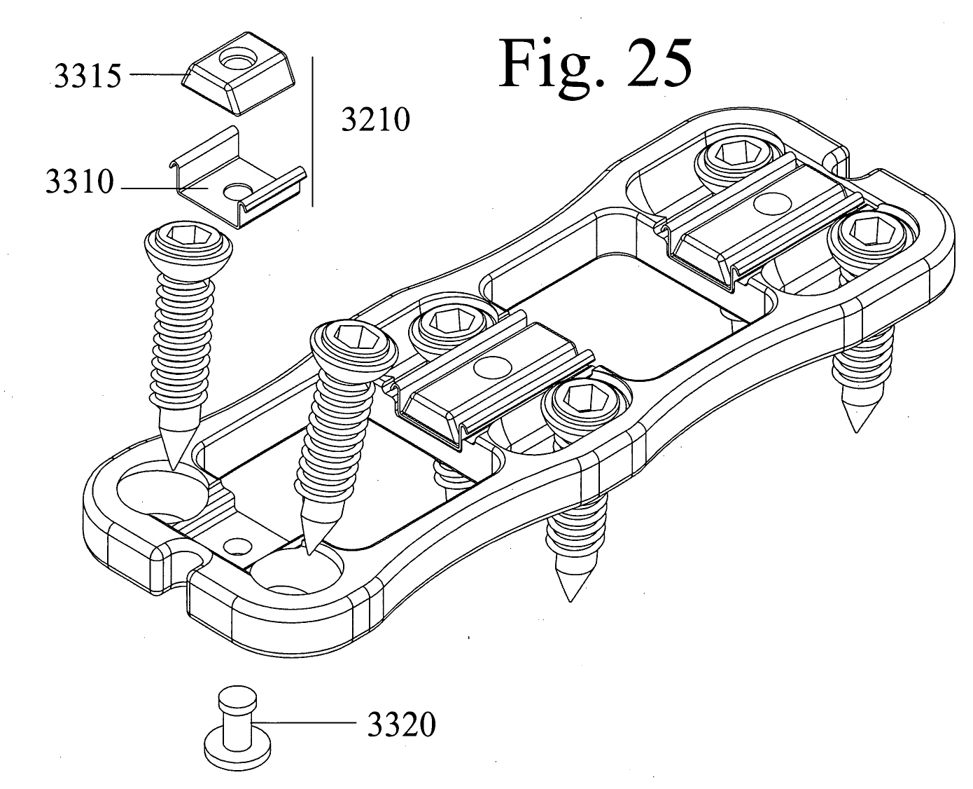

FIGS. 27-29 show alternative self-deploying retaining members 2721 that can be anchored onto the central aspect of a plate and used to retain one or both screws at each vertebral level. A central screw is used to attach each retainer member to the plate's midline. On each side of the retainer is a borehole that is adapted to accept a bone screw. The lateral aspect of a circumferential ring 2729 of each retainer overlies the medial aspect of one borehole on each side of the midline. Advancement of a bone screw through its borehole and into the underlying bone produces medial displacement of that portion of the circumferential retainer ring that overlies the medial aspect of that borehole. After the screw is fully seated within the borehole, the resilient circumferential retainer ring will automatically return to its native, non-displaced position above the medial aspect of the borehole. In this way, the retainer ring will partially cover the screw head and prevent screw back-out. While retainers for the non-slotted bore hole are illustrated, an elongated embodiment can be similarly fashioned to retain bone screws within the slotted bore holes. A bone screw retainer that fits within the plate's bore holes can be employed with this device. Such retaining elements have been described in U.S. Pat. Nos. 5,954,722; 6,331,179; 6,599,290 and others.

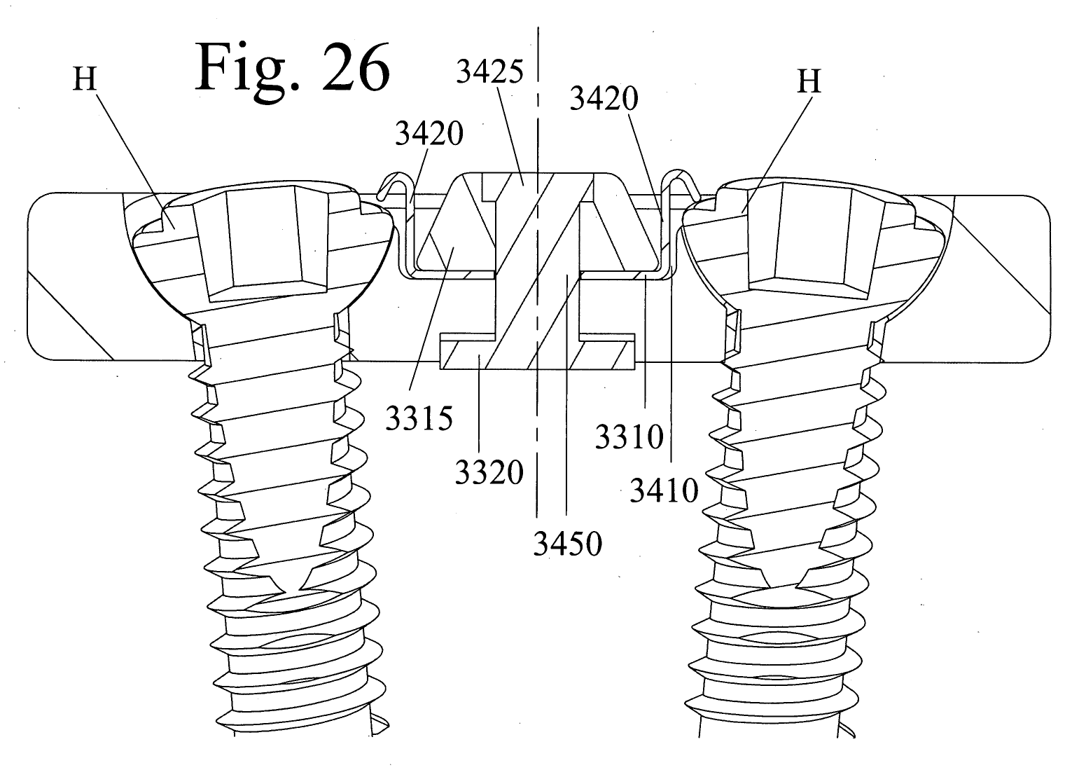

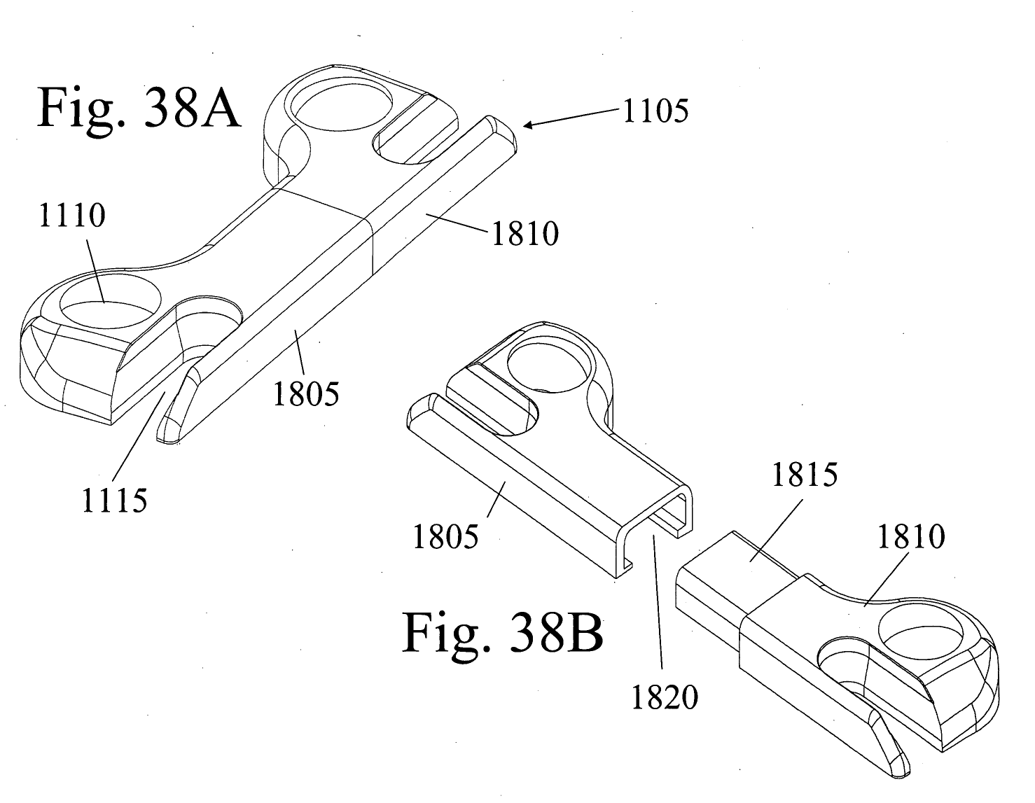

FIG. 30 shows an alternate embodiment of a plate 1105. The plate 1105 is an elongated structure that generally extends along a longitudinal axis. The plate 1105 has a pair of side boreholes 1110 for bone screws. A pair of elongated channels 1115 or apertures are located adjacent to boreholes 1110. The channels or apertures are aligned along a common axis. The boreholes are positioned in enlarged regions that extend laterally outward from the common axis of the channels. The plate can include two enlarged regions as shown in FIG. 30 or can include only a single enlarged region as shown in FIG. 39. Preferably, plate 1105 is implanted with boreholes 1110 on the same side of the vertebral midline as that of the surgical approach. Use of this plate minimizes soft tissue retraction since no screws are implanted on the side opposite to that of the surgical approach. The plate can be positioned on the spine such that at least one hole is one or substantially near the vertebral midline with all remaining holes on one side of the midline. Thus, one or more plate holes are centered on the midline and remainder are placed on only one side of the midline with no holes on the other side of the midline. The remaining holes are preferably on the same side of the midline as that of the surgical approach.

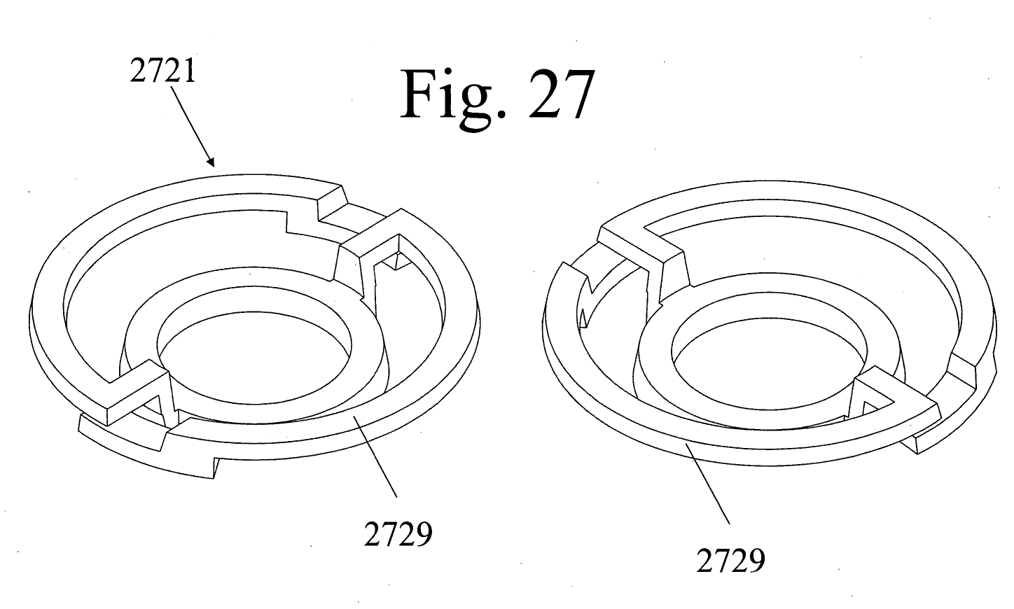

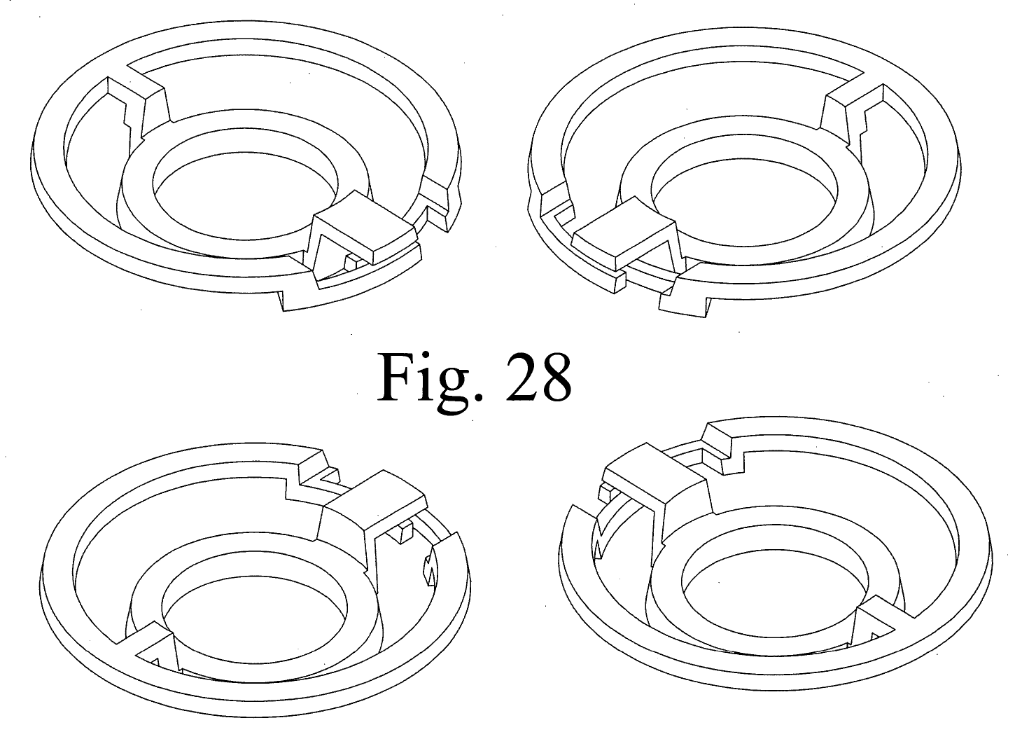

FIGS. 31A and 31B shows perspective views of a modular distraction screw 1210, which is comprised of a distal segment 1220 and a removable proximal segment 1230 coupled to the distal segment 1220. The distal segment 1220 has a head portion 1222 and a threaded shank portion 1224, which can be securely fastened unto a body structure such as bone. The proximal segment 1230 is comprised of an elongated body 1232 that is axially positioned within a sheath-like member 1236. The head portion 1222 fits within a seat 1238 in a distal end of the sheath member 1236.

FIG. 31C shows an enlarged cross-sectional view of the interface between the distal portion segment 1220 and the proximal segment 1230. The distal end of the elongated body 1232 is threaded and engages a threaded bore within the head portion 1222 of the distal segment 1220.

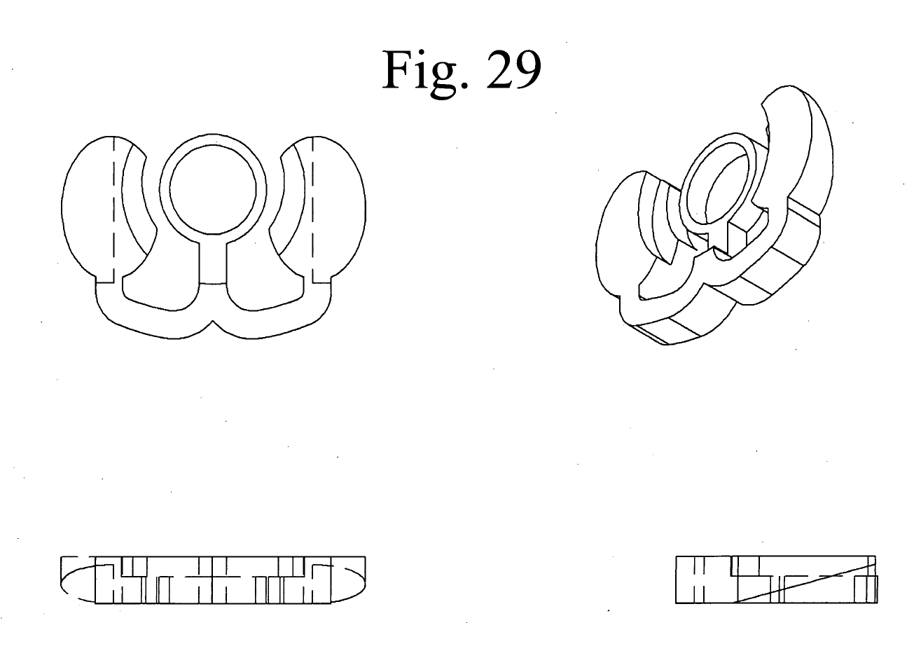

FIGS. 32A-32C show various views of distal segment 1220 of the distraction screw 1210. The distal segment 1220 is comprised of a threaded shank portion 1224 and a head portion 1222. The threads can vary in configuration. For example, the threads can be self-tapping and/or self-drilling. Depending on the particular application, the shank portion 1224 can be of variable lengths and diameter and the threads can be of any design that is suitable for attachment onto bone.

With reference to FIGS. 32A-32C, an embodiment of head portion 1222 is composed of at least two segments, including first segment 1223, which is rotationally positioned within second segment 1225. The second segment 1225 has two or more protrusions that limit the rotation of first segment 1223. When a clockwise rotational force is applied to a central indentation 1221 within first segment 1223, the first segment 1223 will rotate until stopped by the interaction of protrusion 1225 and indentation 1226. Application of additional rotation will cause distal segment 1220 to exert force against the protrusions 1225, such that the entire distal segment turns in unison, such as in a clock-wise fashion. Conversely, application of a counter clock-wise rotational force will return the first segment 1223 to the closed position and further rotation will cause the entire distal segment 1220 to turn in unison in a counter clock-wise fashion.

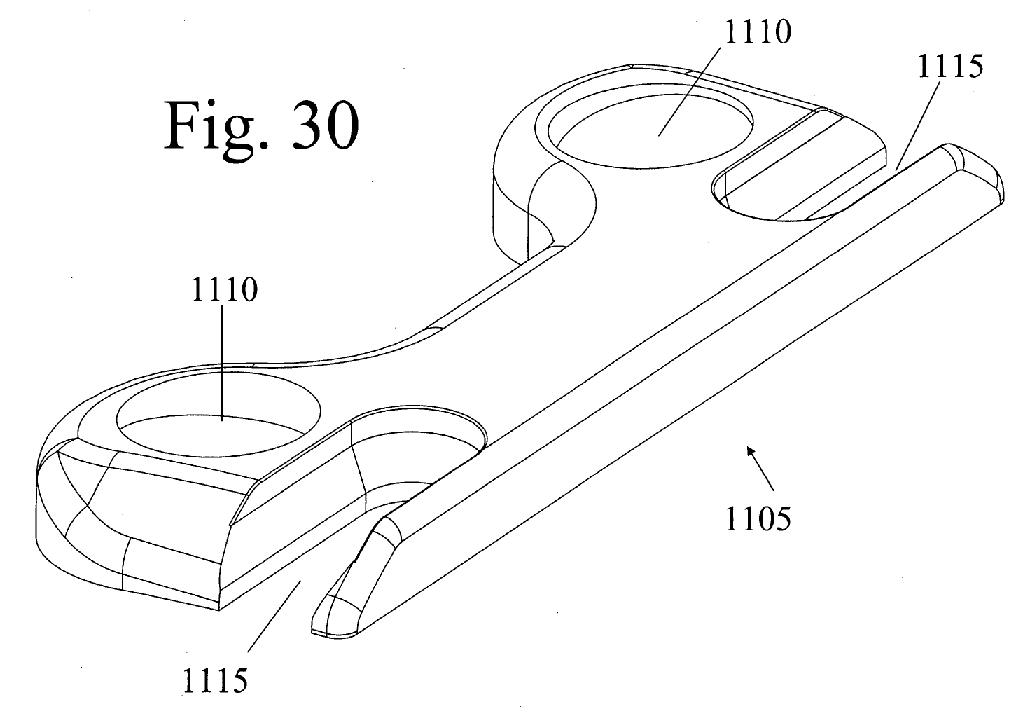

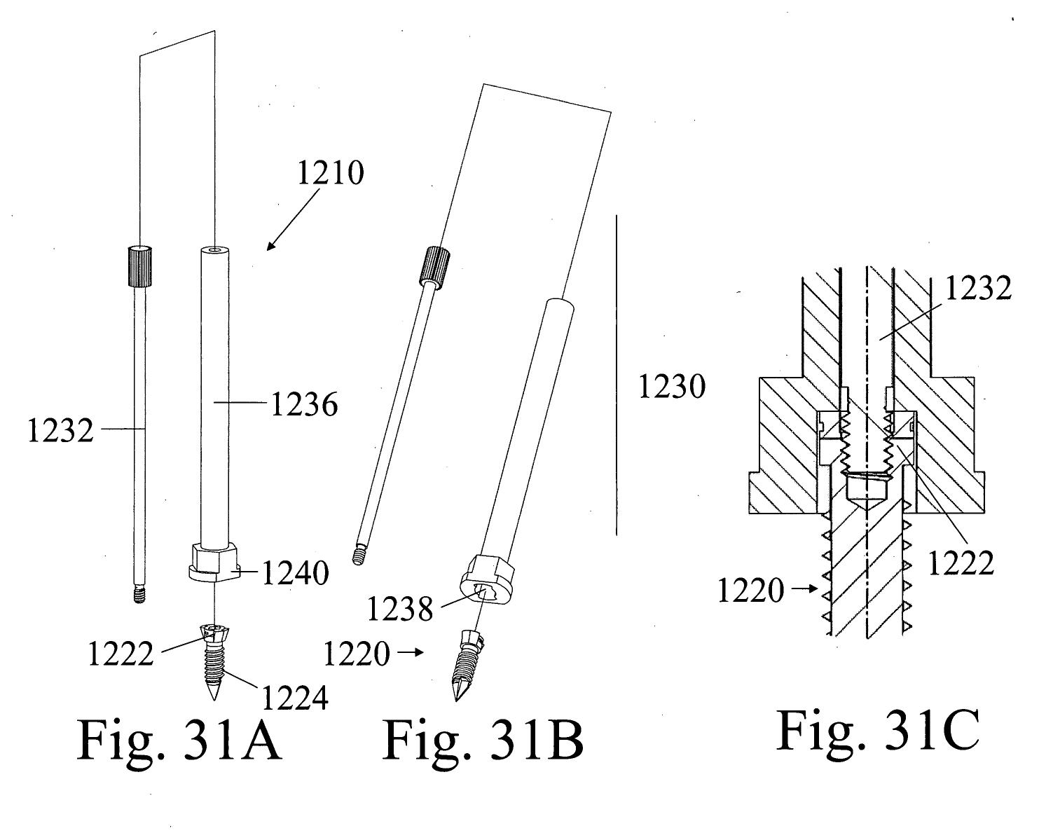

A method of using the distraction screw 1210 is now described. At surgery, the unitary distraction screw is positioned at the vertebral bone surface and a wrench is used to apply a rotational force to a portion 1240 (FIG. 31A) of the elongated body 1236. The applied force causes the entire distraction screw 1210 to rotate in unison so that the thread of the distal segment 1220 engages the underlying bone and the shank 1224 is advanced into the bone. FIG. 33A shows a distraction screw 1210 placed into each of the vertebral bodies V1 and V2 above and below the disc space to be fused. Each distraction screw 1210 is placed with a flat surface (surface B) of the portion 1240 of the elongated body 1236 parallel to the disc space. This ensures that the head 1222 of the distal segment 1220 is oriented with the widest portion perpendicular to the disc space.

After the discetomy and fusion have been performed, each distraction screw is disassembled. FIG. 33B shows the distraction screws 1210 disassembled such that the proximal segments 1230 are detached from the distal segments 1220. The distal segment 1220 remains attached to each vertebral body V1 and V2. The distal segment 1220 provides enhanced structural integrity of the bone by reducing the stress concentration generally expected of an empty opening in a structural member. In addition, leaving the distal segment 1220 attached to bone eliminates the robust bone bleeding encountered after removal of current, commercially-available distraction screws and obviates the need to fill the empty hole with a hemostatic agent.

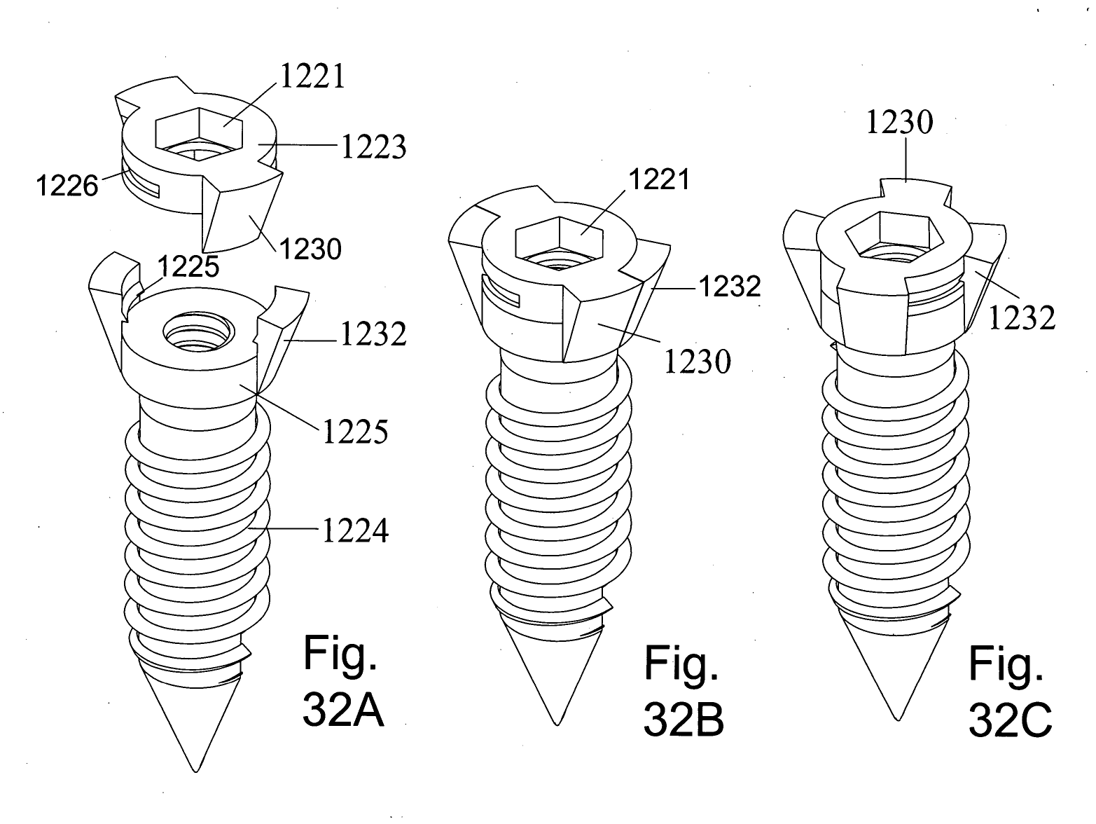

FIG. 34 shows the attached distal segments 1220 with each head 1222 oriented perpendicular to the disc space S. That is, each head 1222 is elongated along an axis that is perpendicular to the plane of the disc space. In this manner, the heads are positioned such that they can be inserted through the channels 1115 of the plate 1105. The distal segments 1220 can be used position and anchor the plate 105 while the bone screws are placed. The distance between the distal segments 1220 is measured and a plate of appropriate size is selected.

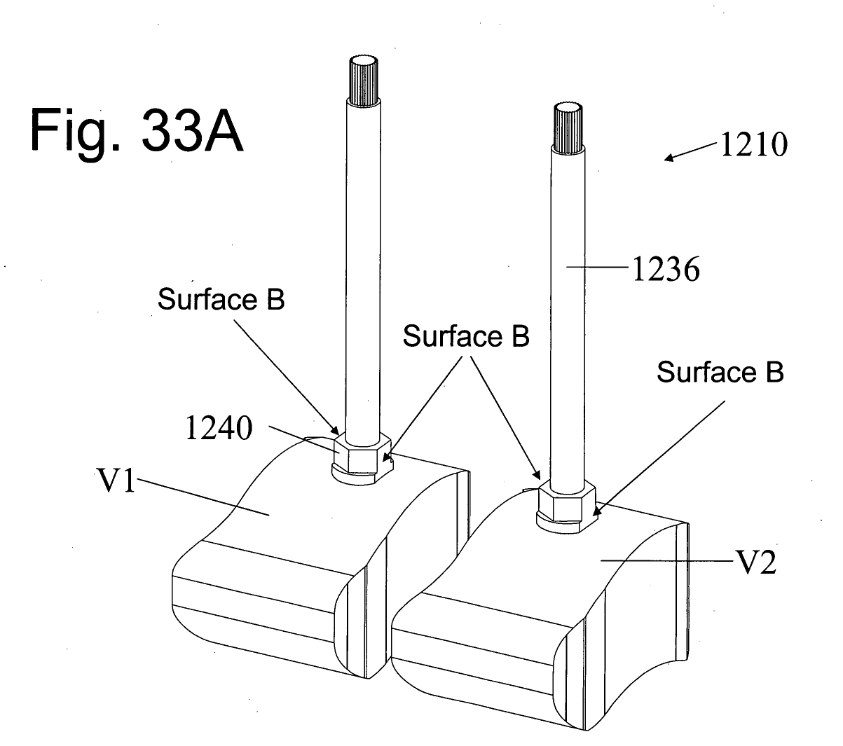

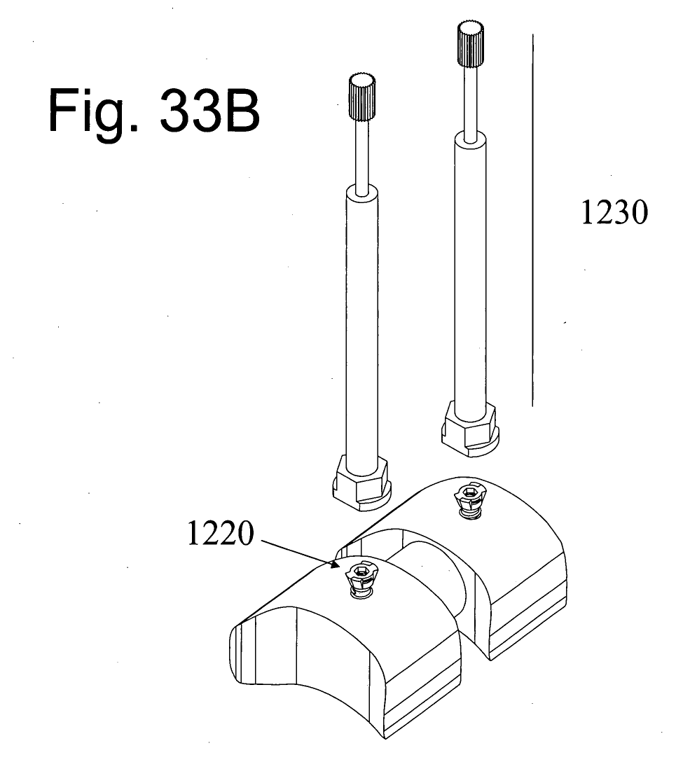

FIG. 35 shows the plate 1105 after is has been lowered onto the distal segments 1220. The plate 1105 is lowered onto the distal segments 1220 such that the distal segments 1220 are positioned within the channels 1115. With the plate 1105 positioned as such, clock-wise rotation is applied to the distal segment 1220 to cause the distal segment 1220 to rotate and drive the shank further into the bone thus immobilizing the plate 1105, as shown in FIG. 36. With the plate 1105 immobilized by the distal segments 1220, one or more bone screws 107 are inserted trough the boreholes in the plate 1105 and used to secure the plate 1105 to the vertebral bodies. FIG. 37 shows the plate 1105 secured to the bone using bone screws 107. It should be appreciated that the previously described retainers can be used with the plate 1105 to retain the screws 107 to the plate 1105. Alternately, any of the other retainer devices that are commonly found in the art may be used. Lastly, one or more of the plate's boreholes 1110 may be slotted.

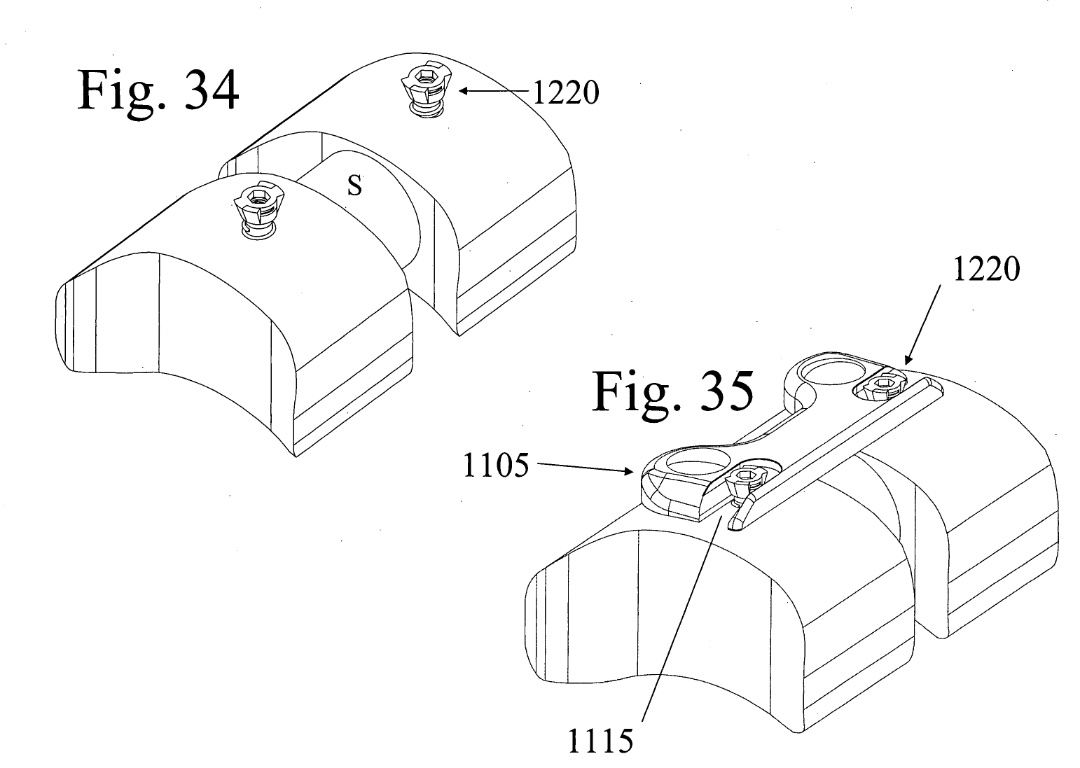

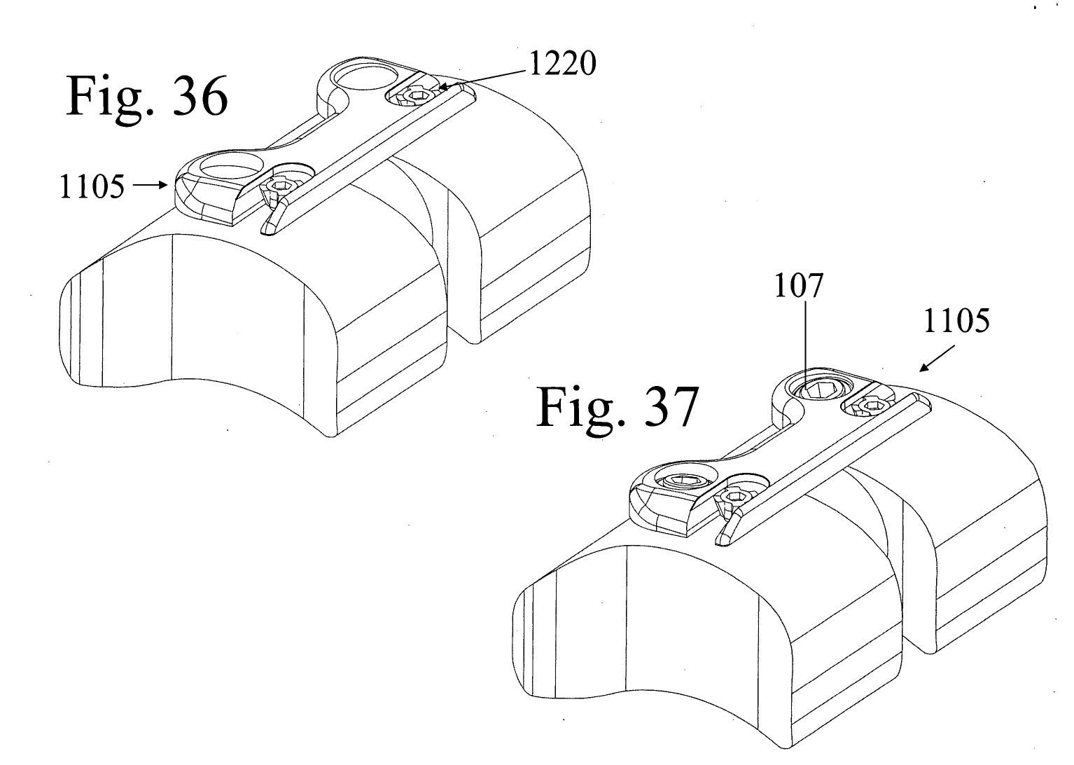

FIG. 38A shows an alternative embodiment of the plate 1105 of FIG. 30. FIG. 38B shows the plate of FIG. 38A in an exploded state. In this embodiment, the plate 1105 includes a first segment 1805 and a second segment 1810 that are movably attached to one another. The plate 1105 includes two or more boreholes 1110 that receive bone screws and elongated channel(s) 1115. The elongated channels 1115 extend generally parallel to a longitudinal axis of the plate 1105. In additional embodiments, the midline channel in either of the plates of FIG. 30 or 38A/B can be oriented in directions other than the longitudinal axis of the plate or the channel can be replaced by a small borehole. Alternatively, the plate may be made with an eccentric borehole and a central spike at each end. The central spike may be driven into the underlying bone to immobilize the plate while the bone screws are placed. In another embodiment, a central bore hole may be used with an off-center spike(s) at each end.

With reference to FIG. 38B, the second segment 1810 includes a protrusion 1815 that slidably fits within a slot 1820 in the first segment 1805. When inserted into the slot 1805, the protrusion 1815 can slide within the slot along the direction of the longitudinal axis of the plate 1105. This permits the first and second segments to move relative to one another even when the segments have been attached to bone. While not illustrated for diagrammatic simplicity, an additional member—such as a pin, threaded fastener, or the like—may be attached onto one segment (for example, 1815) and remain mobile within an aperture located within the second segment (for example, 1805). This feature provides an additional element that can modulate the movement between the two segments—so as to limit the extent of travel, increase the resistance to motion in one or more directions, immobilize the segments in a desired configuration, and the like. Further, an additional feature may be employed that, in one configuration, is stationary relative to a first segment and mobile relative to a second segment while, in a second configuration, it is stationary relative to the second segment and mobile relative to the first segment. This design would provide even more varied and flexible control of the movement between the two segments. Additional movement modulation features may be added as desired.

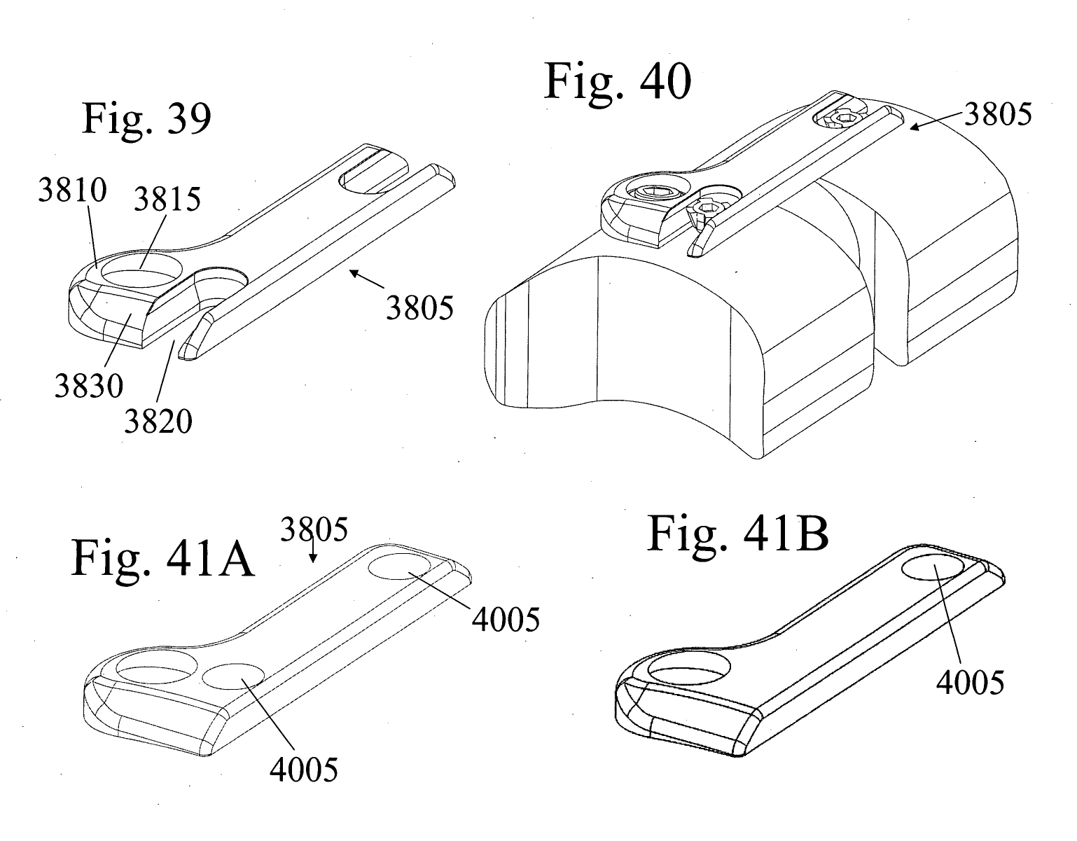

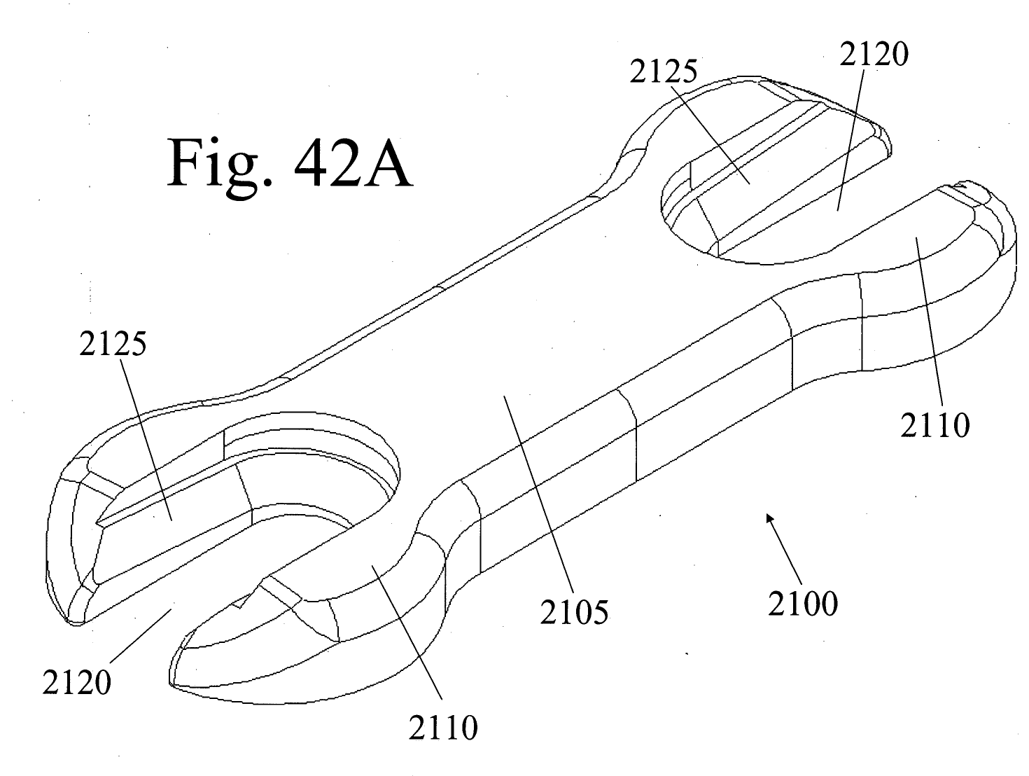

FIG. 39 shows another embodiment of a plate 3805 that is similar to the embodiment shown in FIG. 30. The plate has a widened end region 3810 with a borehole 3815 for receipt of a bone screw. The end region 3810 also includes an elongated, central channel 3820. An opposite end region 3825 includes a second elongated, central channel 3820. The superior and inferior plate edges are tapered at regions 3830. In use, the tapered regions 3830 reduce the ledge-like effect of a non-tapered end and reduce the likelihood that food traveling within the esophagus immediately in front of the plate will be delayed in transit. Thus, the tapered edge design decreases the likelihood of postoperative swallowing difficulties. The plate is preferably implanted with the borehole 3815 on the same side as that of the surgical approach. Since there are no screws on the side opposite to that of the surgical approach, use of this plate minimizes soft tissue retraction. FIG. 40 shows the plate 3805 attached to bone. FIGS. 41A & 41B show another embodiment of a plate 3805 that is shaped similar to the plate of FIG. 40. The plate 3805 in FIG. 41 includes central bore(s) 4005 in place of the channels that are present in the plate of FIG. 40.

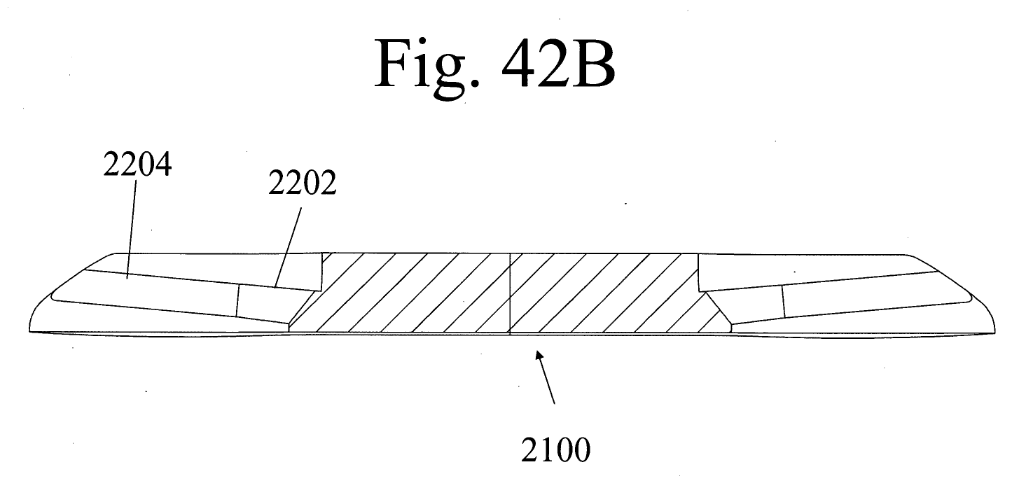

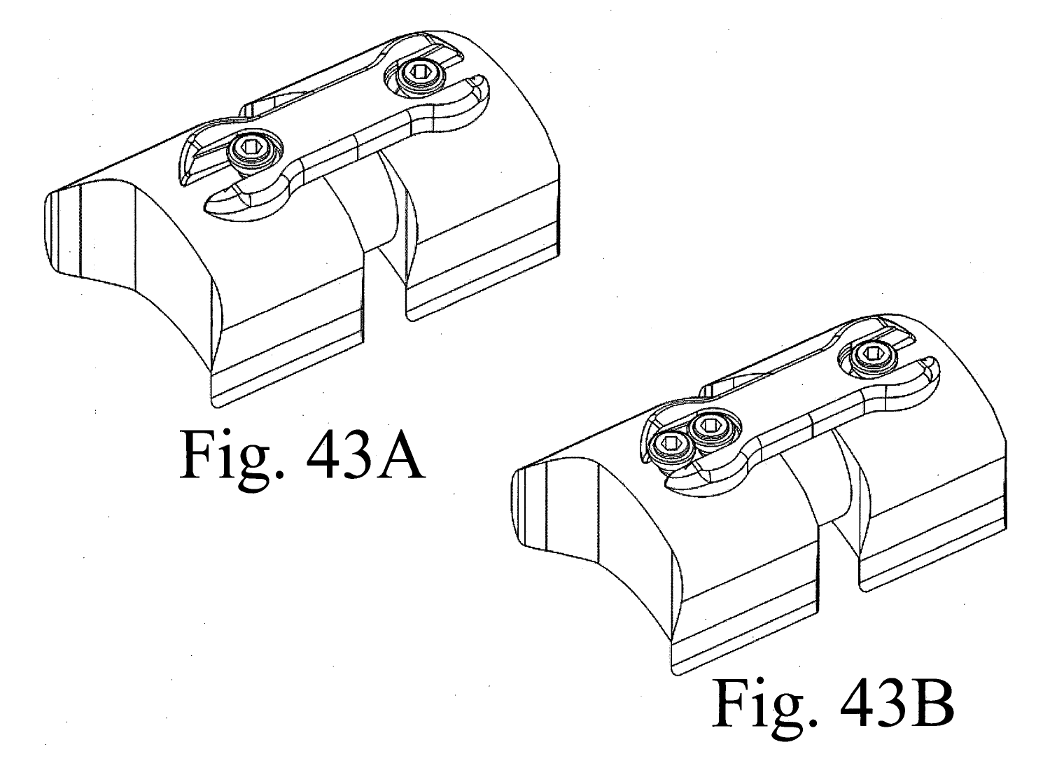

FIG. 42A shows a perspective view of yet another embodiment of a plate 2100. FIG. 42B shows a cross-sectional view of the plate 2100. The plate 2100 includes an elongate central region 2105 and a pair of retaining regions 2110 on opposite ends of the central region 2105. A central, elongated channel 2120 or open-ended slot is positioned in each of the retaining regions 2110. The channels are aligned with a centerline of the plate. The channels include a stepped surface 2125 adapted to engage the head of a bone screw. Any of the disclosed screw retainers or any other type of screw retainer can be used with the plate 2100. The illustrated central channels 2120 are adapted to interact with the multi-segmental distraction screw described herein, although the channels 2120 may be alternatively configured to accommodate any known bone screw design. The slots can be defined by side walls that are angled at a non-perpendicular angle relative to a plane of the base plate.

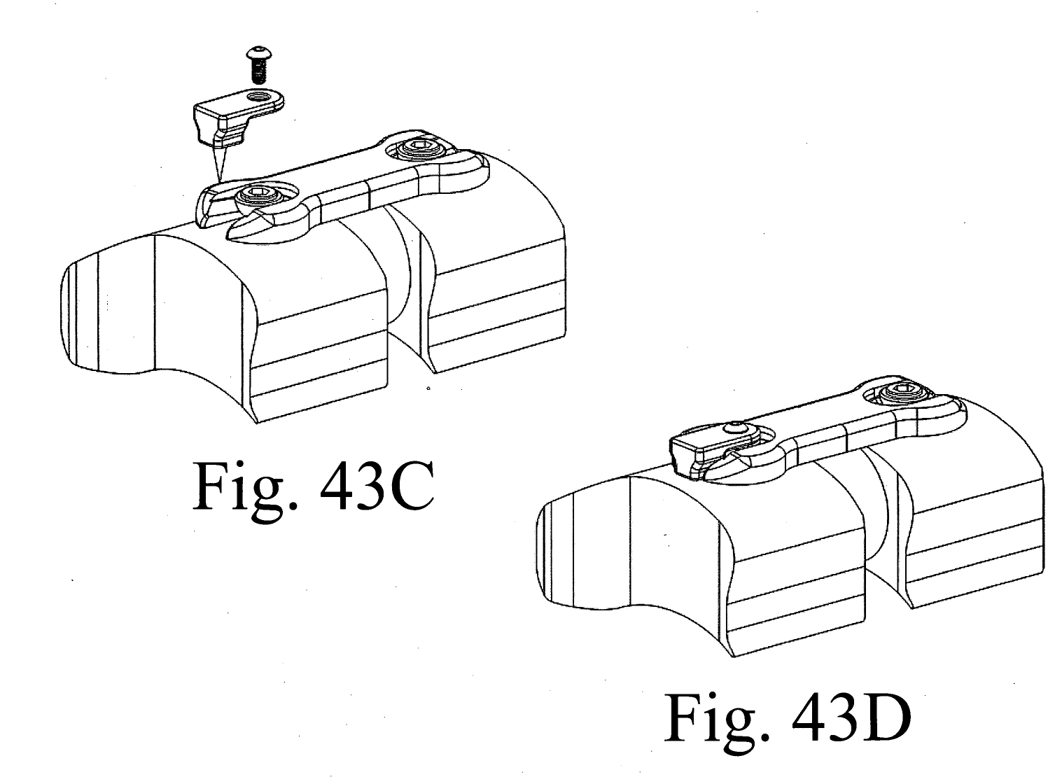

As shown in FIG. 42A and in the cross-sectional view of FIG. 42B, the channels 2120 are defined by sloped walls that gradually deepen as one moves toward the central region 2105. The walls are non-parallel or sloped with respect to one another. In this manner, each of the channels 2120 is angled so that interior end 2202 of the channel is deeper than exterior end 2204 (FIG. 42B). In this way, the vertebral bodies may be compressed towards one another and, once tightened, the screws will maintain the compressive force across the construct. This configuration produces resistance to progressive subsidence that varies with the extent of the subsidence. Any of the channels described herein can have such a wall configuration. To avoid plate rotation relative to the underlying bone (around the screw axis), the inferior aspect of the plate may be fitted with spike(s), ridge(s) and/or textured. As shown in FIGS. 43A and 43B, an additional bone screw may be placed within the slot on one or both ends in order to resist rotation. Alternatively, a screw attachment may be attached onto one or both bone screws—as shown in FIGS. 43C and 43D. The screw attachments may be attached onto the bone screw in the direction of the body of the plate or away from it (as depicted). These features provide an additional point of bone fixation and resist plate rotation. The plate may be positioned on the spine such that the centerline is substantially aligned with a long axis of the spine; and or such that the base plate is positioned on a lateral side of the long axis of the spine.

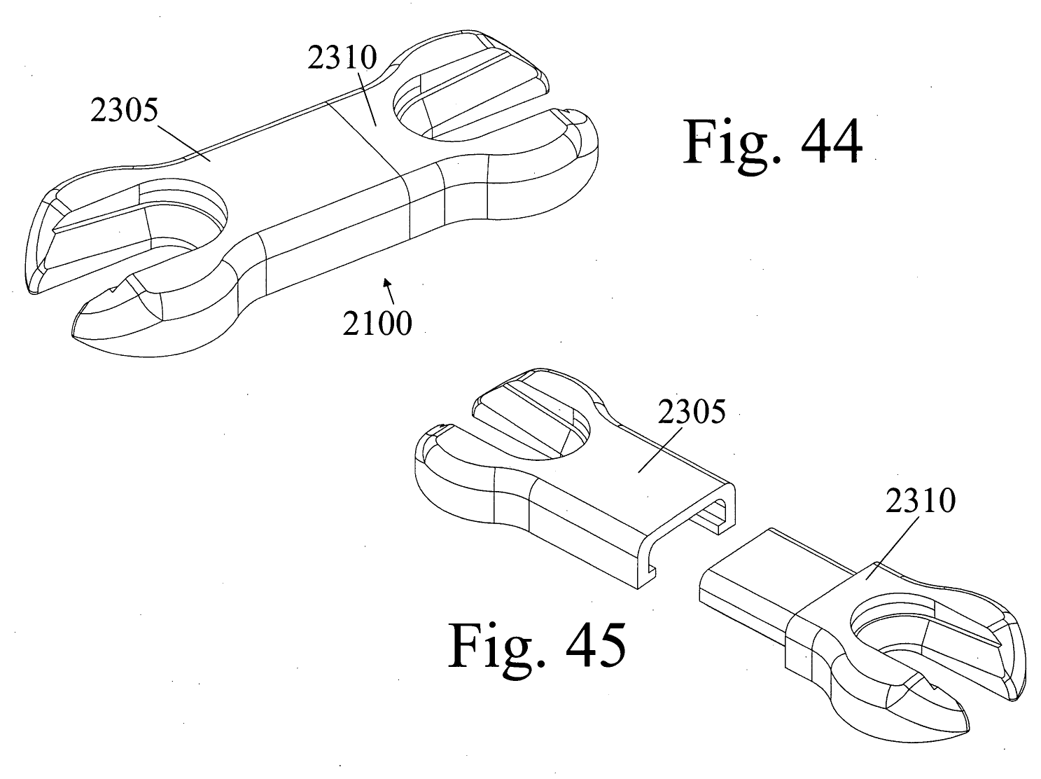

FIGS. 44 and 45 show the plate 2100 formed of two segments 2305 and 2310, which are movably attached to one another as in the embodiment of FIGS. 38A and 38B. While not illustrated for diagrammatic simplicity, an additional member—such as a pin, threaded fastener, or the like—may be attached onto one segment (for example, 2310) and remain mobile within an aperture located within the second segment (for example, 2305). This feature provides an additional element that can modulate the movement between the two segments—so as to limit the extent of travel, increase the resistance to motion in one or more directions, immobilize the segments in a desired configuration, and the like. Further, an additional feature may be employed that, in one configuration, is stationary relative to a first segment and mobile relative to a second segment while, in a second configuration, it is stationary relative to the second segment and mobile relative to the first segment. This design would provide even more varied and flexible control of the movement between the two segments. Additional movement modulation features may be added as desired.

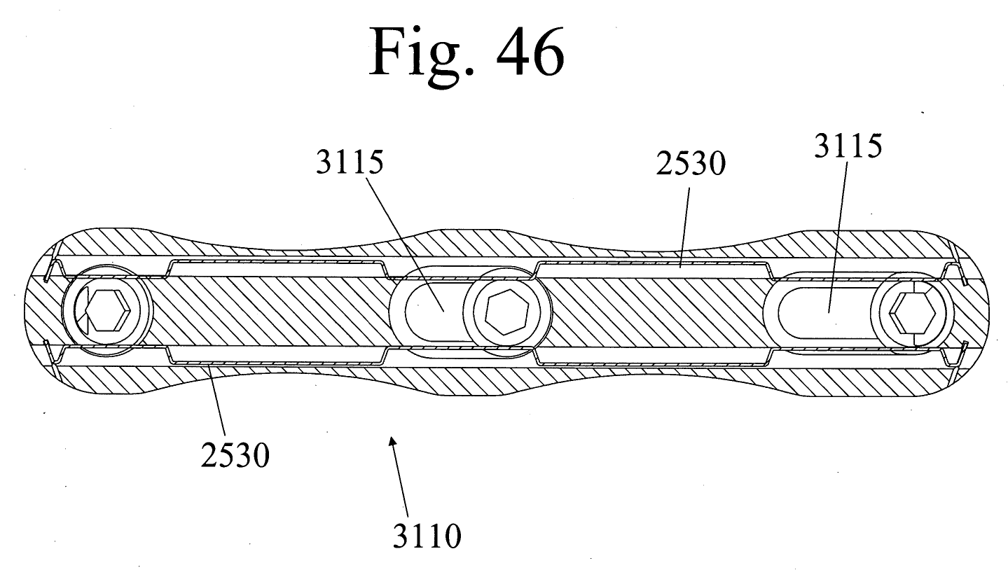

FIG. 46 shows another embodiment of a plate 3110 comprised of an elongated structure having a single line of boreholes 3115 where at least one borehole is an elongated slot. The plate 3110 includes one or more retainer members 2530 that are configured according to the retainer member described above with reference to FIGS. 18-22. In the illustrated embodiment, the retainer members 2530 are positioned such that each borehole 3115 includes a pair of retainer members 2530 on opposite sides of the borehole.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, a surface of any of the implants may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, any assembly or its components can also be entirely or partially made of a shape memory material or other deformable material.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. A bone plate system comprising:
a base plate having at least one aperture for location adjacent to a bone member, the aperture including a seat;
a bone screw sized to be inserted through the aperture such that the bone screw sits within the seat of the aperture for engaging the bone member;
a retainer member adjacent the aperture, the retainer member adapted to transition between a deployed state wherein the retainer member at least partially blocks the aperture for retaining the bone screw in the seat, and an undeployed state wherein the bone screw permits the bone screw to be inserted into the aperture, wherein the retainer member automatically transitions to the undeployed state as the bone screw is inserted into the aperture and automatically transitions to the deployed state once the bone screw is seated within the seat.

2. A bone plate system as in claim 1, wherein the retainer member comprises a clip.

3. A bone plate system as in claim 1, wherein the clip includes a retaining section that attaches to the base plate and a prong with an edge that engages a screw head, the prong adapted to resiliently move into engagement with the screw head.

4. A bone plate system as in claim 1, wherein the retainer member is rod-shaped.

5. A bone plate system as in claim 1, wherein the base plate has at least two apertures, and wherein a single retainer member has a length that permits the single retainer to partially block the at least two apertures.

6. A bone plate system as in claim 1, wherein the retainer member has a size that does not completely surround the perimeter of the borehole.

7. A bone plate system as in claim 1, wherein the aperture is circular.

8. A bone plate system as in claim 1, wherein the aperture is an elongated slot.

9. A bone plate system as in claim 8, wherein the elongated slot has width that tapers moving along the length of the slot.

10. An instrument for placing a bone plate, comprising:
a handle;
at least one grasping member that removably attaches to a bone plate to deliver the plate to an operative field wherein the instrument transitions to a locked state wherein at least a portion of the instrument locks to at least one screw attached to underlying bone.

11. An instrument as in claim 1, wherein grasping member has an aperture sufficiently sized so as to permit passage of a bone screw through the aperture and into a screwhole of the attached bone plate, wherein the aperture aligns with the screwhole when the grasping member is attached to the plate.

12. An instrument as in claim 11, wherein the aperture in the grasping member is sized to permit passage of a drill that forms the bone holes prior to passage of a bone screw through the aperture bone screw placement.

13. An instrument as in claim 11, wherein the instrument includes a pair of arms that move relative to one another so as to lock onto a bone screw or a distraction screw coupled to the plate to thereby immobilize the plate relative to the bone screw or distraction screw.

14. An instrument as in claim 13, wherein the instrument includes an actuator adapted to move the arms.

15. A method of positioning a bone plate, comprising:
attaching a grasping instrument to the plate to immobilize the grasping instrument relative to the plate such that an aperture in the grasping instrument aligns with a screwhole in the plate;
immobilizing the grasping instrument relative to a structure immobilized on the bone to thereby immobilize the plate relative to the bone;
inserting a bone screw through the aperture in the grasping instrument and into the screwhole.

16. A method as in claim 16, further comprising:
inserting a drill through the aperture in the grasping instrument and through the screwhole; and
forming a pathway in the bone using the drill for passage of the screw into the bone.

17. A method as in claim 16, wherein immobilizing the bone screw relative to the plate and the bone comprises screwing a shank portion of the bone screw into the bone such that a head portion of the bone screw immobilizes the plate relative to the bone.

18. A method as in claim 16, wherein the grasping instrument is immobilized relative to a structure comprised of a distraction screw such that the grasping instrument and the plate are immobilized relative to the distraction screw.

19. A method as in claim 18, wherein the distraction screw is positioned within an elongated slot in the plate such that the plate can slide along the elongated slot relative to the distraction screw prior to immobilizing the plate relative to the distraction screw.

20. A bone plate system comprising:
a base plate having a first borehole and a second borehole both positioned adjacent a first edge of the plate, wherein the first borehole is positioned a first distance from the first edge and the second boreholes is positioned a second distance from the first edge and wherein the first distance differs from the second distance;
the base plate also having a third borehole and a fourth borehole both positioned adjacent a second edge of the plate, wherein the third borehole is positioned a third distance from the second edge and the fourth boreholes is positioned a fourth distance from the second edge and wherein the third distance differs from the fourth distance.

21. A bone plate system comprising:
a base plate adapted to be attached to a bone, the base plate having a at least one open-ended slots for receipt of a bone screw, the slot positioned along a centerline of the plate.

22. A plate system as in claim 21, wherein the open ended slot is defined by sloped walls having upper ends that gradually deepen moving toward a central region of the plate.

23. A plate system as in claim 21, wherein the slot is angled so that an interior end of the slot is deeper than an exterior end.

24. A bone plate system as in claim 21, wherein the slot is defined by side walls that are angled at a non-perpendicular angle relative to a plane of the base plate.

25. A bone plate system as in claim 21, wherein the base plate comprises a first section and a second section that are slidably attached to one another.

26. A bone plate system as in claim 21, wherein the slot is sized to receive at least two screws to prevent rotation of the base plate when attached to the bone.

27. A bone plate system as in claim 26, wherein the base plate further includes a borehole for receipt of a bone screw, the borehole positioned along the centerline.

28. A bone plate system comprising:
a base plate adapted to be attached to a bone, the base plate having two or more apertures for receipt of bone screws, the apertures positioned along a centerline of the plate.

29. A bone plate system as in claim 28, wherein at least one of the apertures is elongated along the centerline.

30. A method of placing a bone plate on a spine of a patient, comprising:
a base plate adapted to be attached to a bone, the base plate having a pair of open-ended slots for receipt of bone screws the slots positioned along a centerline of the plate;
placing the base plate on the spine such that the centerline is substantially aligned with a long axis of the spine; and
securing the base plate to the spine.

31. A method as in claim 30, wherein the base plate is positioned on a lateral side of the long axis of the spine.

32. A bone plate system, comprising:
a base plate having at least two apertures aligned along an axis;
at least one enlarged region of the base plate extending laterally outward from the axis, wherein the enlarged region includes at least one borehole for receipt of a bone screw;
a central region of the base plate having a reduced lateral dimension relative to the enlarged region.

33. A bone plate system as in claim 32, wherein the base plate includes two enlarged regions.

34. A bone plate system as in claim 32, wherein at least one borehole is elongated along the axis.

35. A bone plate system as in claim 32, wherein at least one aperture is an open-ended slot.

36. A method of placing a bone plate on a spine, comprising:
providing a plate having multiple screw holes;
positioning the plate on the spine such that at least one hole is on the vertebral midline with all remaining holes on one side of the midline and no holes on the other side of the midline.

37. A method as in claim 36, wherein the remaining holes are on the same side of the midline as that of a surgical approach to the spine.

38. A method as in claim 36, further comprising inserting a bone screw into the at least one hole on the vertebral midline.
